(19) United States
(12) Patent Application Publication
(10) Pub. No.: US 2011/0295152 A1
(43) Pub. Date: Dec. 1, 2011

(54) PUNCTURE METHOD, INCISION NEEDLE, PUNCTURE NEEDLE, NEEDLE CAP AND INSTRUMENT FOR BLOOD PURIFICATION TREATMENT

(75) Inventors: Masatomi SASAKI, Tokyo (JP); Toru SHINZATO, Toyohashi-shi (JP); Yasuyo MARUYAMA, Toyohashi-shi (JP); Yukie SHIONOYA, Hoi-gun (JP)

(73) Assignees: NEXTIER CORPORATION, Aichi (JP); ASAHI KASEI KURARAY MEDICAL CO., LTD., Tokyo (JP)

(21) Appl. No.: 13/115,275

(22) Filed: May 25, 2011

(30) Foreign Application Priority Data
May 25, 2010 (JP) 2010-119634
Jun. 2, 2010 (JP) 2010-126867
Jun. 2, 2010 (JP) 2010-126885

(51) Int. Cl. A61B 5/15; A61B 17/00; A61M 37/00

(52) U.S. Cl. 600/573; 604/22; 606/108

(57) ABSTRACT

The present invention is a method of puncturing a shunt blood vessel, using a incision needle having a pointed extremity that protrudes apically from a hypothetical angled plane formed by cutting straight through the tip of the needle at an angle, wherein the pointed extremity of the incision needle is inserted into a shunt blood vessel, thereby forming a reverse U-shaped incision on the surface of the shunt blood vessel, with the apex of the incision being on the bottom side in the direction of insertion.

### BACKGROUND OF THE INVENTION

1. Field of the Invention

The present invention relates to a method of puncturing a surface of a shunt blood vessel, to a tubular or cylindrical incision needle for forming an incision on the surface of the shunt blood vessel, to a puncture needle for puncturing a skin and penetrating the shunt blood vessel through a puncture hole formed by incision in the surface of the shunt blood vessel, to a needle cap for removing a scab at the entrance of a puncture route on the skin surface, and to an instrument for blood purification treatment.

2. Description of the Related Art

In hemodialysis and other forms of blood purification for example, a needle is pierced through the patient's skin into a shunt blood vessel, and blood is removed and returned through this needle. This puncture operation is required every time blood purification is performed, and is very painful for the patient. In order to reduce the pain of puncture, therefore, a method called buttonhole puncture has arisen in recent years, in which an ordinary sharp needle is used during the initial puncture operation to form puncture route 102 through skin 100 of the body to shunt blood vessel 101 as shown in FIG. 19 for example, and then in subsequent puncture operations a less-sharp dull needle is inserted through puncture route 102 to puncture shunt blood vessel 101 (Japanese Patent Application Laid-open No. 2009-045124). In this method, the patient's pain is reduced because no new holes need to be opened in skin 100 once the puncture route is formed.

Needle 104 used in the initial puncture operation has angled end face 104a, which is angled toward the tip as shown in FIG. 20, and pointed extremity 104b formed at the tip of angled end face 104a. When shunt blood vessel 101 is punctured with needle 104, needle 104 is first inserted at an angle into skin 100 with angled end face 104a facing upward, and then pushed in the direction of shunt blood vessel 101. After pointed extremity 104b reaches the surface of shunt blood vessel 101, the needle is inserted into shunt blood vessel 101 with the same orientation.

### SUMMARY OF THE INVENTION

When needle 104 is inserted into shunt blood vessel 101, puncture hole 101a, which consists of a U-shaped incision opening towards the far side with the bottom of the incision on the front side in the direction of insertion, is formed on the surface of shunt blood vessel 101 as shown in FIG. 21. In the second and subsequent puncture operations, a dull needle is inserted into shunt blood vessel 101 through the incision forming this puncture hole 101a.

After formation of puncture hole 101a consisting of a U-shaped incision as described above, dull needle 105 is inserted into puncture route 102 formed by passage of needle 104 during the initial puncture operation as shown in FIG. 22, and once the pointed extremity of dull needle 105 reaches the surface of shunt blood vessel 101, the tip of dull needle 105 is used to find the position on puncture hole 101a at which the puncture hole can be opened with the weakest pressing force, or in other words optimal press point 101b.

However, in the case of a puncture hole 101a formed with a conventional needle 104, optimal press point 101b is near the bottom of the U-shaped incision that forms puncture hole 101a. Thus, the position of optimal press point 101b of puncture hole 101a, which must be found with the tip of dull needle 105 after formation of puncture hole 101a, is on the front side with respect to insertion direction X of dull needle 105.

Thus, when searching for optimal press point 101b of insertion hole 101a with the tip of dull needle 105, the tip of dull needle 105 is slid from the front side towards the far side in insertion direction X, but if the tip of dull needle 105 does not immediately find optimal press point 101b on the front side, it then moves away from optimal press point 101b, so that ultimately the tip of dull needle 105 is unable to find optimal press point 101b. Even if the tip of dull needle 105 is able to find optimal press point 101b immediately, moreover, because optimal press point 101b of puncture hole 101a is on the front side, dull needle 105 must then be stood up perpendicular to the shunt blood vessel surface in order to insert the tip of dull needle 105 into puncture hole 101a. Not only does this apply excess force to puncture route 102, but often the position of the tip of dull needle 105 is slightly shifted with respect to optimal press point 101b, so that more time is required to insert dull needle 105 into puncture hole 101a, or the shunt blood vessel is damaged, causing pain to the patient.

It is an object of the present invention, which was developed in light of these issues, to make it possible for the optimal press point to be found easily during the second and subsequent punctures, and for the needle to be inserted more smoothly into the shunt blood vessel.

While the tip of ordinary needle 210 is formed with pointed cutting face 210a as shown in FIG. 32, the tip of the dull needle 220 shown in FIG. 33 has angled end face 220a formed by cutting the end of a tube at an angle, and has a rounded tip.

In the buttonhole puncture method described above, puncture hole 201a formed by the initial puncture operation on the surface of shunt blood vessel 201 is formed as a U-shaped incision as shown in FIG. 34, so that in the second and subsequent puncture operations, dull needle 220 is inserted into the shunt vessel starting from puncture start point 204 of puncture hole 201a. Specifically, in the second and subsequent puncture operations the tip of dull needle 220 is aligned with this puncture start point 204, and pressure is applied to open puncture hole 201a appropriately and insert dull needle 220 into shunt blood vessel 201. In aligning the tip of dull needle 220 with puncture start point 204, the operator relies on the sense of touch to find puncture start point 204 with the tip of dull needle 220.

However, it is hard to find the shunt blood vessel with the dull needle 220 described above because the needle does not readily transmit the sensation of the blood vessel to the fingers. As a result, dull needle 220 may have to be inserted multiple times into puncture route 202 for example, or it may take time to find puncture start point 204 of the shunt blood vessel, adding time to the puncture operation. Moreover, if the tip of dull needle 220 makes contact with the shunt blood vessel at a point even slightly removed from puncture start point 204, more force is required to insert the tip of dull needle 220 into the shunt blood vessel, sometimes causing damage to the shunt blood vessel and pain to the patient for example. Experience and skill are required to find puncture start point 204 with the tip of dull needle 220 so that the tip of dull needle 220 can be inserted quickly and with little force into the shunt blood vessel.

It is an object of the present invention, which was developed in light of these matters, to improve the ability of a needle to find the puncture start point when puncturing a shunt blood vessel via a puncture route.

In the aforementioned buttonhole puncture method, in the second and subsequent puncture operations, a scab formed at the entrance to the puncture route is removed, and the dull needle is inserted into the puncture route through this entrance. Skin damage is likely because the conventional way of removing the scab formed at the entrance to the puncture route is to detach the scab with the tip of the needle, and when the dull needle is used as the needle, hygiene problems arise such as contamination of the needle tip. It has therefore been proposed that the needle cap that covers the dull needle before use be provided with a pointed extremity at the tip of the cap, which can then be used to remove the scab (Japanese Patent Publication 2009-542343).

However, because this pointed extremity at the tip of the needle cap is formed extending forwards in the lengthwise direction, the fine pointed extremity of the needle cap is inserted between the skin and the scab for example when removing a scab formed at the entrance to the puncture route, and the needle cap must then be moved forwards in the lengthwise direction in order to lift the scab. In this method, however, the scab is difficult to see because the operator's line of sight is blocked by the operator's fingers or by the needle cap itself, making the operation more difficult. This makes the scab removal operation more time-consuming, and the skin may also be damaged if the pointed extremity of the needle cap contacts the wrong area. Moreover, because the pointed extremity of the needle cap is moved back and forth in order to detach the scab, the needle cap is likely to rub against the skin and cause skin damage.

It is an object of the present invention, which was developed in light of these matters, to facilitate the operation of scab removal so that a scab can be removed without damage to the skin.

To achieve the aforementioned objects, the present invention is a method of puncturing a shunt blood vessel, having a step of puncturing a shunt blood vessel with a tubular or cylindrical incision needle and thereby forming a incision on the surface of a shunt blood vessel, wherein the incision needle has an angled end face at the tip of the needle, and the tip of the angled end face has a pointed extremity that protrudes apically from a hypothetical angled plane formed by cutting straight through the tip of the needle at an angle, and wherein the step of forming the incision has a step of arranging the incision needle so that the angled end face faces the skin, a step of puncturing the skin with the pointed extremity of the incision needle and inserting the needle toward the shunt blood vessel to thereby form a puncture route, and a step of inserting the pointed extremity of the incision needle into the shunt blood vessel to thereby form a reverse U-shaped incision that opens towards the surface, with the apex of the incision being on the bottom side in the direction of insertion.

In the present invention, a incision opening towards the front side in a reverse U shape with the starting point on the far side is formed on the surface of the shunt blood vessel, and this reverse U-shaped incision functions as the site where the dull needle enters the shunt blood vessel, or in other words as the puncture hole. In a puncture hole of this kind, the optimal site for the dull needle to contact the puncture hole is on the far side with respect to the insertion direction of the dull needle. As a result, it is easier to find the optimal press point with the tip of the dull needle when the tip of the dull needle is slid from the front side to the far side in the dull needle insertion direction in order to find the optimal press point of the puncture hole during second and subsequent punctures after the incision is created. Once the tip of the dull needle finds the optimal press point, moreover, it is not necessary to stand the dull needle up perpendicular to the surface of the shunt blood vessel or to push the puncture hole strongly with the dull needle because the optimal press point of the puncture hole is located on the far side in the insertion direction. As a result, insertion of the dull needle into the puncture hole is not time-consuming, and does not cause damage to the shunt blood vessel or pain to the patient.

In this puncture formation method, the incision needle may be one in which the pointed extremity is bent toward the central axis of the needle. In this case, puncture of the shunt blood vessel can be accomplished accurately with the angled end face of the incision needle facing downwards.

The incision needle may also be one having a cutting face formed on at least a part of the angled end face that includes the pointed extremity. In this case, puncture of the shunt blood vessel can be performed more smoothly with the angled end face of the incision needle facing downwards.

The incision needle may be one having such a cutting face extending from the pointed extremity to the angled surface of the angled end face.

The incision needle may be one having a reverse cutting face on the reverse surface of the angled end face. This allows the shunt blood vessel to be punctured more smoothly with the angled end face of the puncture needle facing downwards.

The incision needle may be one having such a reverse cutting face on at least the pointed extremity.

The incision needle may be one having such reverse cutting faces on both sides of the reverse surface of the angled end face posterior to the pointed extremity.

The incision needle may be one having another pointed extremity extending apically behind the pointed extremity of the angled end face.

The incision needle may be one having a groove on the reverse surface for accepting blood of the shunt blood vessel from the tip of the angled end face.

After formation of the reverse U-shaped incision described above, the aforementioned puncture method may also include a step of penetrating the puncture route in the skin with a puncture needle, and inserting the needle into the shunt blood vessel through the aforementioned incision.

In this puncture method, the puncture needle may have a tubular part with an angled end face angled with respect to the needle axis and a protruding part that protrudes forwards from the tip of the angled end face of this tubular part, wherein the tip of the protruding part is narrower than the tubular part, and is curved convexly outwards.

Because this invention has a protruding part that protrudes forward from the tip of the angled end face of the tubular part and is narrower at the tip than the tubular part, the sensation of the shunt blood vessel is communicated more easily to the fingers by this protruding part, thereby improving the ability of the needle to find the puncture start point. This facilitates and expedites the operation of puncturing the shunt blood vessel. Moreover, damage to the shunt blood vessel and pain to the patient from the tip of the protruding part are prevented because the tip of the protruding part is curved convexly in front.

When the dull needle is inserted into the shunt blood vessel cavity, meanwhile, because the puncture hole is generally gummed shut with fibrin, the blood vessel wall must be pushed until it opens. The tip of a conventional dull needle pushes down on the blood vessel wall in accordance with the large curvature radius of the angled end face, so that a longer part of the dull needle tip contacts the blood vessel wall, which tends to disperse the insertion force. In the present invention, because the protruding part is narrow, the part of the tip that contacts the blood vessel wall is short, and the insertion force is more concentrated.

The curvature radius of the tip of the protruding part of the puncture needle may also be smaller than the curvature radius of the tip of the angled end face. In this case, the sensation of the shunt blood vessel in contact with the tip of the protruding part is communicated more easily to the fingers, thereby improving the ability of the needle to find the puncture start point on the shunt blood vessel.

The angle of inclination of the angled end face relative to the protruding direction of the protruding part of the puncture needle may be less than 60°. In this case, less force is required to puncture the shunt blood vessel with the needle because the resistance of the needle is less during puncture.

This puncture method may also include a step of removing a scab formed at the entrance of the puncture route on the skin surface with a needle cap of the puncture needle before inserting the puncture needle into the puncture route.

In this puncture method, the needle cap has a hollow holding part for holding the needle and a scab removal part at the tip of the holding part, and the scab removal part is formed from a circular arc-shaped outer surface curving around the insertion axis of the needle and an inner surface located inside the outer surface, and may also have a sharp top edge that is formed from this outer surface and inner surface, and has a width in the direction of the needle insertion axis.

With this invention, because a sharp top edge is formed at the tip of the needle cap with a width in the direction of the needle insertion axis, a scab can be removed by rotating this top edge around the insertion axis of the needle. Thus, the contact area between the top edge and the scab is easier to see because the operator's field of view is not obstructed by the needle cap itself or by the fingers of the operator removing the scab. Because the top edge for removing the scab has a width, moreover, it is easy to align the top edge with the scab. This facilitates the operation of removing the scab, which can thus be removed without damage to the skin.

This scab removal part is formed substantially in a crescent shape in cross-section, and in this crescent cross-section, the angle formed by the arc of the outer surface and the arc of the inner surface at the top edge may be 5 to 89°. If the angle is 89° or less the scab removal part is easy to insert between the scab and the skin, while if it is 5° or more the scab removal part can maintain its strength, making it easy to peel off the scab.

A protruding part with a flat anterior surface protruding forwards on the outer side of the top edge can be formed at the tip of the needle cap. In order to maintain the sterile condition of the needle with the needle cap before use, it is stored and transported in a sealed sterile bag. With the present invention, because a protruding point with a flat anterior surface is formed at the tip of the needle cap, the tip of the needle cap is prevented from opening a hole in the sterile bag during transport for example. The needle can thus be transported appropriately. Moreover, because the anterior surface of the tip of the needle cap is flat, the strength of the needle cap is improved with respect to collisions with other objects.

This protruding part of the needle cap can be formed from the base end of the tip towards the front on the right-left center line as seen from the front of the tip, while the outer surface of the scab removal part extends outwards around the circumference of the tip from the protruding part, with the top edge formed at the periphery of this outer surface away from the protruding part. The tip can be stronger in this case because the protruding part is formed on the center line, passing between the left and right, of the tip of the needle cap. This means that the scab removal part can be thinner, and the angle of the top edge can be sharper. This facilitates the operation of scab removal.

The scab removal part can also be formed on both sides of the protruding part. In this case, scab removal can be performed from either the right or the left, allowing the scab to be removed from the side with best visibility.

The scab removal part of the needle cap can be formed on both sides of the right-left center line as seen from the front of the tip, and the top edges on both sides can be formed revere to each other on the circumference of the tip. In this case, scab removal can be performed from either the right or the left, allowing the scab to be removed from the side with best visibility.

The inner surface of the needle cap may also be curved convexly towards the outer surface. This facilitates scab removal because the angle of the top edge can be sharper.

The needle cap may also have a scab removal part formed at the tip in such a way that the scab removal part has a flat outer face parallel to the needle insertion axis and an inner surface located inside this outer face, so that a sharp top edge part is formed by this outer face and inner surface with a width in the direction of the needle insertion axis.

With this invention, because a sharp top edge is formed at the tip of the needle cap with a width in the direction of the needle insertion axis, the scab can be removed by rotating the top edge around the insertion axis. Thus, the contact area between the top edge and the scab is easier to see because the operator's field of view is not obstructed by the needle cap itself or by the fingers of the operator removing the scab. Because the top edge for removing the scab has a width, moreover, it is easy to align the top edge with the scab. This facilitates the operation of removing the scab, which can thus be removed without damage to the skin.

A protruding part with a flat anterior surface protruding forwards from the top edge can be formed at the tip of the needle cap.

This protruding part of the needle cap can be formed from the base end of the tip towards the front on the right-left center line as seen from the front of the tip, while the outer face of the scab removal part is formed beside the protruding part of the needle cap, with the top edge is formed at the periphery of this outer face furthest from the protruding part.

This scab removal part can also be formed on both the right and left sides of the protruding part of the needle cap.

This scab removal part can be formed on both left and right sides of the center line as seen from the front of the tip, and the top edges on both sides can be formed reverse to each other to the left and right of the protruding part.

The inner surface of the needle cap may also be curved convexly toward the outer surface.

The inner surface of this scab removal part may also be angled in the direction of the needle insertion axis so that the scab removal part grows thinner as it nears the anterior surface. This gives the scab removal part a thick part and a thin part, so that the scab can be removed with the part that is most suited to the size and shape of the scab.

This needle cap can also be used for a needle for puncturing during blood purification treatment.

This needle cap can also be used for a needle for puncturing during drug administration or blood withdrawal.

Another aspect of the present invention is a method of puncturing a shunt blood vessel with a puncture needle through a puncture hole formed by incision in the surface of the shunt blood vessel, using a puncture needle comprising a tubular part having an angled end face angled relative to the needle axis and a protruding part that protrudes forward in a linearly from the tip of the angled end face of this tubular part, wherein the tip of the protruding part is narrower than the tubular part, and is curved convexly in the front, and the method of puncturing comprises a step of inserting the puncture needle in the shunt blood vessel through the puncture hole formed by incision starting with the protruding part.

Another aspect of the present invention is a method of puncturing a shunt blood vessel, comprising a step of removing a scab formed at an entrance to a puncture route on the skin surface with a needle cap to be mounted on a puncture needle, and a step of puncturing the shunt blood vessel with the puncture needle after removal of the scab, wherein the needle cap has a hollow holding part for holding the needle and a scab removal part at the tip of the holding part, and the scab removal part is formed from a circular arc-shaped outer surface curving around the insertion axis of the needle and an inner surface located inside this outer surface, and has a sharp top edge that is formed from this outer surface and inner surface with a width in the direction of the needle insertion axis, and the scab is removed with the scab removal part of this needle cap.

Another aspect of the present invention is a tubular or cylindrical incision needle for puncturing a shunt blood vessel and forming an incision on the surface of the shunt blood vessel, having an angled end face at the tip of the needle, wherein the tip of the angled end face has a pointed extremity that protrudes apically from a hypothetical angled plane formed by cutting straight through the tip of the needle at an angle, and wherein the pointed extremity is bent towards the central axis of the needle, cutting faces are formed on at least a part of the angled end face that includes the pointed extremity, reverse cutting faces are formed on at least a part of the reverse surface of the angled end face that includes the pointed extremity, and the foremost edge of the pointed extremity is formed by the aforementioned cutting faces arranged symmetrically with the foremost edge, while the reverse cutting faces are disposed not at the foremost edge, but arranged symmetrically with the center of the pointed extremity therebetween.

In this incision needle, the distance between the tip of the pointed extremity and the outer surface of the needle body can be 6% to 50% of the outer diameter of the needle body. A distance of 6% or more makes it easier for the incision needle to break into the skin, while 50% or less allows for smooth insertion under the skin.

Another aspect of the present invention is a puncture needle for puncturing a shunt blood vessel through a puncture hole formed by incision in the surface of the shunt blood vessel, comprising a tubular part having an angled end face angled relative to the needle axis and a protruding part that protrudes forwards from the tip of the angled end face of the tubular part, wherein the tip of the protruding part is narrower than the tubular part, and is curved convexly in front, and wherein the radius of the protruding part is 200 μm or more. In this case, damage to the skin and shunt blood vessel wall can be controlled and the needle can be inserted smoothly with little pain because the tip of the protruding part is rounded with a radius of 200 μm or more.

The curvature radius Re of the tip of the protruding part and the radius Ro of the outer diameter of the tube can be in the relationship of 0.1×Ro≦Re<1.0×Ro. If the curvature radius of the tip of the protruding part satisfies 0.1×Ro≦Re in this way, damage to the surface of the shunt blood vessel can be controlled, while if Re<1.0×Ro, the puncture force can be concentrated, enhancing the ability of the needle to find the puncture start point. When the curved surface of the tip of the protruding part comprises multiple curvature radii, the smallest curvature radius is given as curvature radius Re.

Another aspect of the present invention is a needle cap for removing a scab at the entrance of a puncture route on the skin surface, comprising a hollow holding part for holding a needle and a scab removal part at the tip of the holding part, wherein the scab removal part is formed from a circular arc-shaped outer surface curving around the insertion axis of the needle and an inner surface located inside the outer surface, and has a sharp top edge that is formed from this outer surface and inner surface and has a width in the direction of the needle insertion axis, and wherein the scab removal part is formed substantially in a crescent shape in cross-section, and the angle formed by the arc of the outer surface and the arc of the inner surface at the top edge in this crescent cross-section is 5° to 89°. With such an angle of 5° to 89°, the scab removal part is easy to insert between the scab and the skin, but the scab removal part can still maintain its strength, making it easy to detach the scab.

The length of the arc of the outer surface of the crescent cross-section may be 5% to 50% of the maximum outer diameter of the holding part. If it is more than 5%, the strength of the scab removal part is enhanced, thus facilitating the operation of removing the scab. Damage to the packaging material during product transport can also be controlled in this way. On the other hand, an angle of 50% or less is economical, and also allows twisting of the needle cap to be minimized during the operation of applying the scab removal part to the scab and the skin surface.

Another aspect of the present invention is a blood purification treatment instrument capable of constituting part of a blood purification circuit, and comprising the aforementioned needle cap, a needle inserted in this needle cap, a tube with the needle connected to the tip thereof, and a connecting part for connecting the posterior end of this tube to another tube.

Thus, damage to a shunt blood vessel and pain to the patient can be prevented with the present invention because after a reverse U-shaped incision is formed on the surface of the shunt blood vessel and a puncture route is prepared, a dull needle or other needle can be inserted easily and with less effort into the shunt blood vessel via this reverse U-shaped incision. Also, in the present invention it is easy to find a puncture start point at the bottom of the reverse U-shaped incision on the surface of the shunt blood vessel with a needle when inserting a needle into the puncture route and then puncturing and inserting a dull needle or other needle into the shunt blood vessel via the reverse U-shaped incision. The puncture operation can thus be accomplished rapidly and easily. Damage to the shunt blood vessel and pain to the patient from the needle can also be prevented. The operation can also be performed appropriately regardless of experience or skill. The operation of scab removal is also easy with the present invention, and a scab can be removed without damage to the skin.

### BRIEF DESCRIPTION OF THE DRAWINGS

FIG. 1 shows an outline of a blood purification treatment instrument;

FIG. 2 is a top view of the tip of a incision needle;

FIG. 3 is a side view of the tip of a incision needle;

FIG. 4 is a reverse view of the tip of a incision needle;

FIG. 5 is an explanatory drawing showing a incision needle before insertion into the skin;

FIG. 6 is an explanatory drawing showing a incision needle inserted into the skin;

FIG. 7 is an explanatory drawing showing the puncture hole and optimal press point of a shunt blood vessel;

FIG. 8 is an explanatory drawing showing a incision needle puncturing a shunt blood vessel;

FIG. 9 is an explanatory drawing showing a dull needle inserted into a puncture route;

FIG. 10 is a top view of the tip of another incision needle in which the reverse cutting faces are in a different location;

FIG. 11 is a side view of the tip of another incision needle;

FIG. 12 is a reverse view of the tip of another incision needle;

FIG. 13 is a reverse view of a incision needle with a groove;

FIG. 14 is a top view of the tip of a cylindrical incision needle;

FIG. 15 is a side view of the tip of a cylindrical incision needle;

FIG. 16 is a top view of the tip of a incision needle having another pointed extremity;

FIG. 17 is a side view of the pointed extremity of a incision needle having another pointed extremity;

FIG. 18 shows an outline of an indwelling needle;

FIG. 19 is an explanatory drawing showing a puncture route formed under the skin;

FIG. 20 is an explanatory drawing showing a conventional needle;

FIG. 21 is an explanatory drawing showing a puncture hole and optimal press point formed on a shunt blood vessel with a conventional needle;

FIG. 22 is an explanatory drawing showing a dull needle inserted into a conventional puncture route;

FIG. 23 shows an outline of the blood purification treatment instrument of the 2^{nd} embodiment;

FIG. 24 is an oblique view of the tip of a dull needle;

FIG. 25 is a top view of the tip of a dull needle;

FIG. 26 is a side view of the tip of a dull needle;

FIG. 27 is an explanatory drawing showing a dull needle before insertion into the skin;

FIG. 28 is an explanatory drawing showing a dull needle inserted into the skin;

FIG. 29 is an explanatory drawing showing a dull needle puncturing a shunt blood vessel;

FIG. 30 is a side view of the tip of a dull needle with a gradation on the angled end face;

FIG. 31 is an explanatory drawing showing a puncture route formed under the skin;

FIG. 32 is an explanatory drawing of an ordinary puncture needle;

FIG. 33 is an explanatory drawing of a conventional dull needle;

FIG. 34 is an explanatory drawing showing the puncture hole and puncture start point on a shunt blood vessel;

FIG. 35 shows an outline of the blood purification treatment instrument of the 3^{rd} embodiment;

FIG. 36A is a side view of a needle cap;

FIG. 36B is a front view of the tip of a needle cap as seen from the front;

FIG. 37 shows an outline of a blood purification circuit;

FIG. 38 shows a scab being removed with a needle cap;

FIG. 39 shows a scab being removed with the tip of a needle cap;

FIG. 40 is a front view showing the tip of needle cap with a scab forming part on only one side, as seen from the front;

FIG. 41 is a front view showing the tip of a needle cap having an inner surface composed of inclined planes, as seen from the front;

FIG. 42A is a side view of a needle cap having a protruding part;

FIG. 42B is a top view of a needle cap having a protruding part;

FIG. 43 is a front view of the tip of a needle cap having a protruding part, as seen from the front;

FIG. 44A is a side view of a needle cap in which the inner surface is tapered towards the front;

FIG. 44B is a front view of the tip of a needle cap in which the inner surface is tapered towards the front;

FIG. 45A is a side view of a needle cap having another scab removal part;

FIG. 45B is a front view of the tip of a needle cap having another scab removal part;

FIG. 46 is a front view of a needle cap showing a scab removal part having a curved inner surface;

FIG. 47 is a front view of a needle cap having scab removal parts on the right and left;

FIG. 48 is a front view of a needle cap showing a scab removal part having a protruding part;

FIG. 49 is a side view of a scab removal tool; and

FIG. 50 is a top view of a scab removal tool.

### DETAILED DESCRIPTION OF THE INVENTION

Preferred embodiments of the present invention are explained below with reference to the drawings. In this Description and in the drawings, components having effectively the same function are assigned the same symbols, and redundant explanations are omitted.

### 1^{st} Embodiment

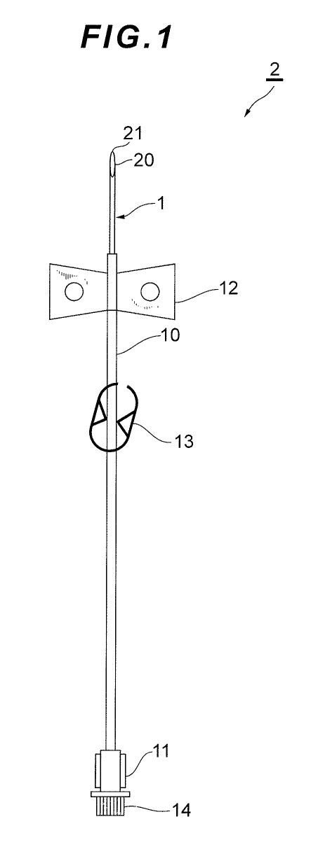

FIG. 1 is an explanatory drawing showing one example of blood purification treatment instrument 2 having incision needle (shunt blood vessel surface incision-forming instrument) 1 of this embodiment.

Blood purification treatment instrument 2 has tubular incision needle 1 for forming a incision on the surface of a shunt blood vessel of a patient during blood purification treatment for example, tube 10 with incision needle 1 connected to the tip thereof, and connecting part 11 for connecting the posterior end of tube 10 to another tube. The part of tube 10 near incision needle 1 is provided with holding part 12, which the operator holds when moving incision needle 1. Clamp 13 is also attached to tube 10. Cap 14 fits onto connecting part 11.

Blood purification treatment instrument 2 is connected to another tube by means of connecting part 11 for example to thereby constitute part of a blood purification circuit having a hemopurification device (not shown). Blood purification treatment instruments 2 are attached to the end section on the blood removal side and the end section on the blood return side of the blood purification circuit, and remove and return blood via incision needles 1, which can also serve as puncture needles during blood purification treatment. Blood purification treatment includes for example hemodialysis, plasma exchange, plasma adsorption, blood component elimination and the like.

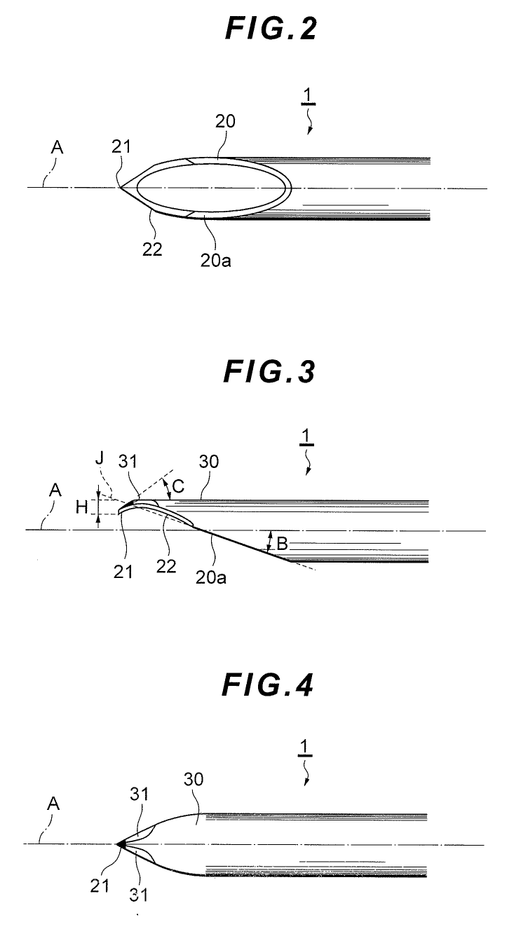

As shown in FIGS. 2, 3 and 4, the tip of incision needle 1 has angled end face 20, which is angled with respect to central axis A of the needle. This inclination angle B (shown in FIG. 3) is preferably about 5° to 21°. Pointed extremity 21 is formed at the tip of angled end face 20. Pointed extremity 21 protrudes apically from hypothetical angled plane J (an extension of the part of angled end face 20 that defines inclination angle B), which cuts straight through the tip of the needle at an angle. Pointed extremity 21 is bent towards central axis A of the needle. This bend may be a curve or a bend. The bend angle C (shown in FIG. 3) of pointed extremity 21 in the direction of central axis A is preferably about 2° to 90°, or more preferably 8° to 60°. As shown in FIG. 3, the distance H between the position of the tip of pointed extremity 21 and the outer surface of the main body of the needle is equal to 6% to 50% of the outer diameter of the main body of the needle.

Cutting face 22 is formed on angled end face 20. Cutting face 22 is formed by cutting both edges in the horizontal direction (perpendicular to central axis A). Cutting face 22 is formed for example along the periphery of angled face 20a from pointed extremity 21 to near the center of angled end face 20, where the transverse diameter of angled face 20a (part having inclination angle B) is greatest. Cutting face 22 may also be formed to a point further back (further up the angled face) from the position where the transverse diameter of angled face 20a is the greatest.

Reverse cutting faces 31 are formed on reverse surface 30 of angled end face 20. Reverse cutting faces 31 extend backwards for example from pointed extremity 21 along both edges of reverse face 30. Reverse cutting faces 31 are formed shorter than cutting face 22 for example.

The process of puncturing a shunt blood vessel during blood purification treatment using a incision needle 1 configured as described above is explained next.

First, in the initial blood purification treatment, blood purification treatment instrument 2 having incision needle 1 is removed from its sterile package, and connected to a blood purification circuit.

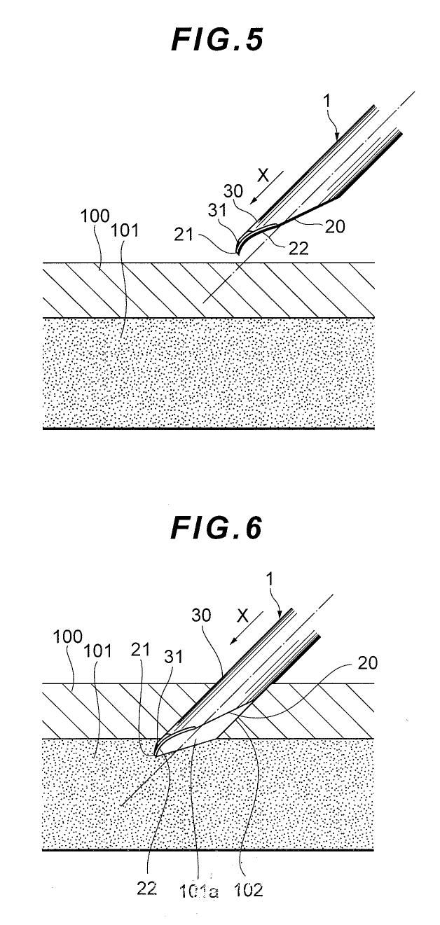

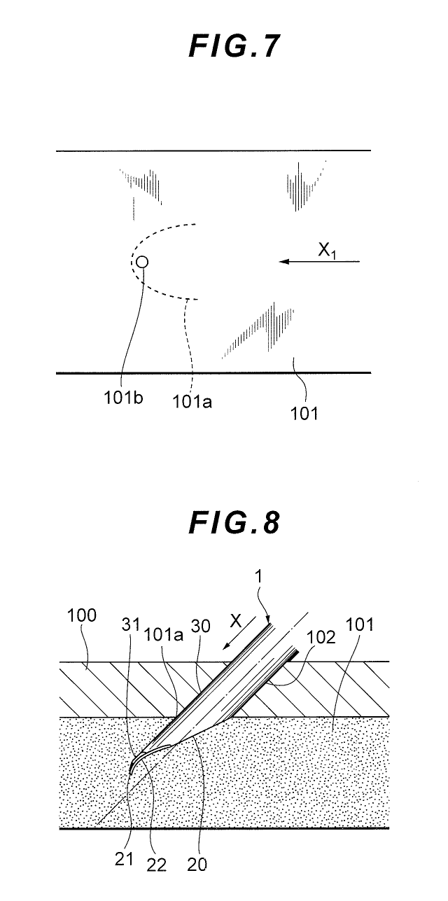

Next, as shown in FIG. 5, incision needle 1 is disposed with pointed extremity 21 on top and angled end face 20 facing down, or in other words with angled end face 20 facing skin 100, and inserted at a specific angle from diagonally above the surface of skin 100. Incision needle 1 is thus inserted from skin 100 under the skin towards shunt blood vessel 101, forming puncture route 102. When pointed extremity 21 of incision needle 1 reaches the surface of shunt blood vessel 101 as shown in FIG. 6, it opens puncture hole 101a to form a incision, and enters shunt blood vessel 101 with pointed extremity 21 first. As shown in FIG. 7, puncture hole 101a (incision) splits open in a rough reverse U-shape starting from the far side of the incision relative to horizontal direction component X1 of insertion direction X of the incision needle, and optimal press point 101b is formed on the far side of the puncture hole 101a. Next, as shown in FIG. 8, incision needle 1 is advanced further so that all of angled end face 20 of incision needle 1 is inserted into shunt blood vessel 101.

After this, blood is removed and returned via incision needle 1 to perform blood purification treatment. When blood purification treatment is complete, incision needle 1 is pulled out. Puncture route 102 is thus formed in skin 100 extending to shunt blood vessel 101, and puncture hole 101a (incision) is formed on the surface of shunt blood vessel 101.

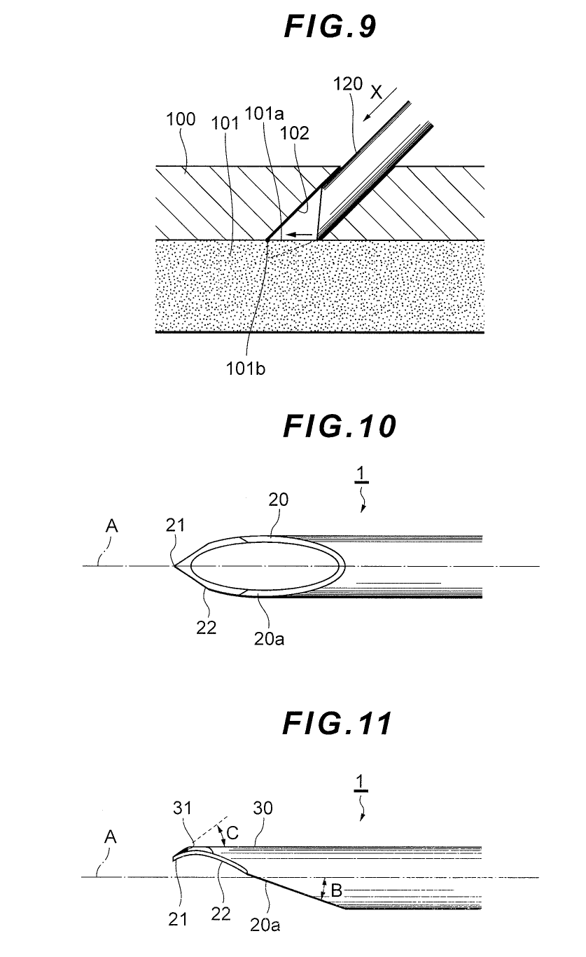

In blood purification treatment after the incision is prepared, dull needle 105 is used to puncture shunt blood vessel 101. First, the scab formed at the entrance of puncture route 102 is removed. Next, as shown in FIG. 9, dull needle 105 is inserted into puncture route 102, and when dull needle 105 reaches the surface of shunt blood vessel 101, it searches for optimal press point 101b. Force is exerted in insertion direction X on dull needle 105, which slides from the front side to the far side, and reaches optimal press point 101b on the far side of the puncture incision. Once dull needle 105 reaches optimal press point 101b, puncture hole 101a opens starting from optimal press point 101b, and dull needle 105 enters shunt blood vessel 101 from puncture hole 101a. Blood is then removed and returned via dull needle 105 to perform blood purification treatment.

In the embodiment described above, pointed extremity 21 of angled end face 20 is bent towards central axis A of the needle. As a result, the angle between pointed extremity 21 and the surface of shunt blood vessel 101 can be adequately secured when incision needle 1 is used to puncture shunt blood vessel 101 with angled end face 20 facing down and pointed extremity 21 on top. Thus, shunt blood vessel 101 can be punctured accurately with incision needle 1 even in so-called upside-down puncturing with pointed extremity 21 on top.

In upside-down puncturing, optimal press point 101b is formed on the far side of puncture hole 101a in shunt blood vessel 101 relative to diagonal insertion direction X of incision needle 1. This makes optimal press point 101b easier to find because dull needle 105 is conducted to optimal press point 101b on the far side of the puncture hole by the force of insertion of dull needle 105 after the puncture route is prepared. Because optimal press point 101b is on the far side, moreover, when opening puncture hole 101a with the tip of dull needle 105 after sliding dull needle 105 as far as optimal press point 101b, it is not necessary to stand dull needle 105 up perpendicular to the surface of the shunt blood vessel or to push puncture hole 101a strongly with the tip of dull needle 105 in order to insert dull needle 105 into puncture hole 101a. As a result, less time is required to insert dull needle 105 into puncture hole 101a, and it is possible to prevent damage to the shunt blood vessel and pain to the patient.

In the embodiment described above, smooth upside-down puncturing by incision needle 1 with pointed extremity 21 on top is possible because cutting face 22 is formed on angled end face 20 from pointed extremity 21 to the middle of angled face 20a of angled end face 20.

Moreover, smooth so-called upside-down puncturing by incision needle 1 with pointed extremity 21 on top is also possible because reverse cutting faces 31 are formed on pointed extremity 21 on reverse surface 30 of angled end face 20.

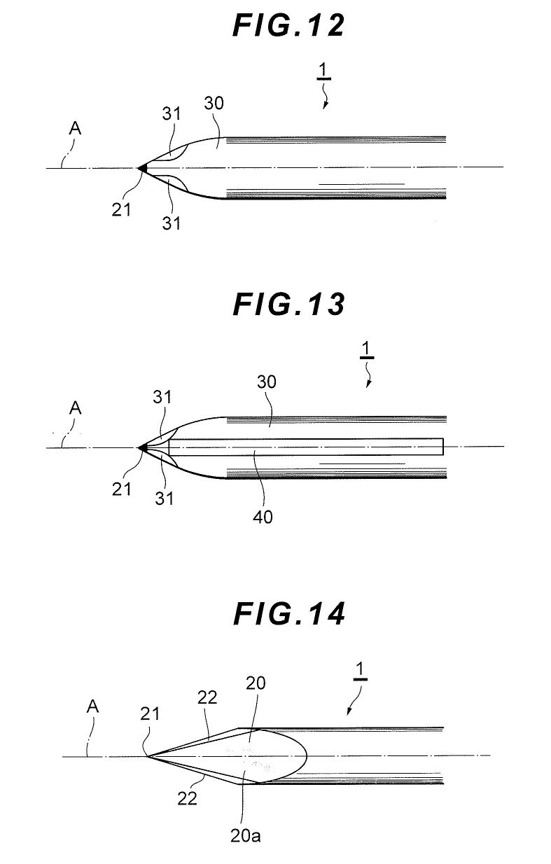

In the embodiment described above reverse cutting faces 31 were formed on pointed extremity 21, but as shown in FIGS. 10, 11 and 12, they may also be formed on both sides of reverse surface 30 of angled end face 20 posterior to pointed extremity 21. This allows for smoother puncturing with incision needle 1 with pointed extremity 21 on top. Reverse cutting faces 31 may also be formed both near pointed extremity 21 and behind pointed extremity 21.

In the incision needle 1 described in the above embodiment, groove 40 for accepting blood of shunt blood vessel 101 from the tip of angled end face 20 can be formed on reverse surface 30 of incision needle 1 as shown in FIG. 13. In this case, groove 40 is formed in a straight line extending backwards from near pointed extremity 21 for example. Groove 40 is preferably about 0.1 mm to 2 mm wide for example and about 15 μm to 500 μm deep. It is thus possible to check visually whether incision needle 1 has entered shunt blood vessel 101 after forming the incision, since blood enters groove 40 by capillary action once angled end face 20 of incision needle 1 is inside shunt blood vessel 101.

A preferred embodiment (1^{st} embodiment) of the present invention was explained above with reference to the attached drawings, but the present invention is not limited to this example. Of course a person skilled in the art could conceive of various changes and modifications within the scope of the concepts described in the Claims, and these are naturally understood to fall within the technical scope of the present invention.

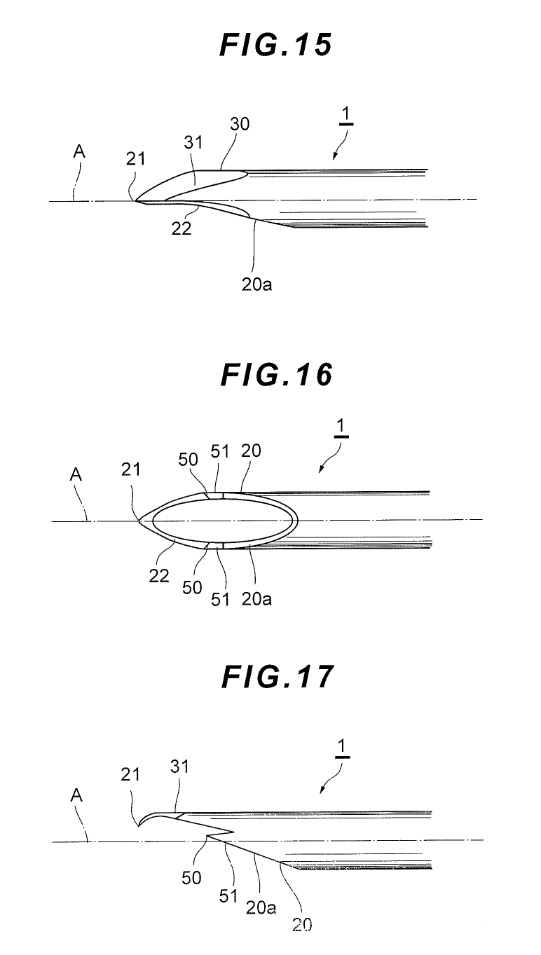

For example, pointed extremity 21 of the incision needle 1 described in the above embodiment can be in another shape as long as it is bent towards central axis A of the needle. Moreover, the shapes, positions and numbers of cutting faces 22 and reverse cutting faces 31 are not limited. Also, the incision needle 1 of the above embodiment had a tubular shape to allow passage of blood inside the needle, but as shown in FIGS. 14 and 15, it can also have a solid cylindrical shape that does not allow passage of blood. A non-tubular needle has increased puncture resistance due to the increased area during puncture, but is advantageous for manufacturing purposes because it is easy to work. This incision needle 1 also forms puncture route 102. In this case, incision needle 1 is inserted into shunt blood vessel 101 in upside-down puncture mode during the initial blood purification operation, forming puncture hole 101a and puncture route 102, while in subsequent blood purification operations dull needle 105 is inserted into shunt blood vessel 101 via puncture route 102 and puncture hole 101a, and blood is removed and returned via this dull needle 105 to perform blood purification treatment.

In the incision needle 1 described in the above embodiment, another apically projecting pointed extremity 50 may also be formed behind pointed extremity 21 of angled end face 20 as shown in FIGS. 16 and 17. The other pointed extremity 50 is formed with a sharp angle of about 30° for example as seen from the side. Cutting face 51 is formed on the upper surface of the other pointed extremity 50. In general, the sharper the inclination angle of cutting face 22, the greater the puncture ability, but this makes the angled end face longer, so that the angled needle face projects beyond the shunt blood vessel width, increasing the risk of blood leakage. With a two-stage structure such as that shown in FIGS. 16 and 17, the puncture ability can be improved by means of a sharper cutting face, while providing a incision needle with little risk of blood leakage.

In the above embodiment, incision needle 1 is used during blood purification treatment, but for example the present invention is also applicable to an incision needle for use to administer drug or to take blood sample.

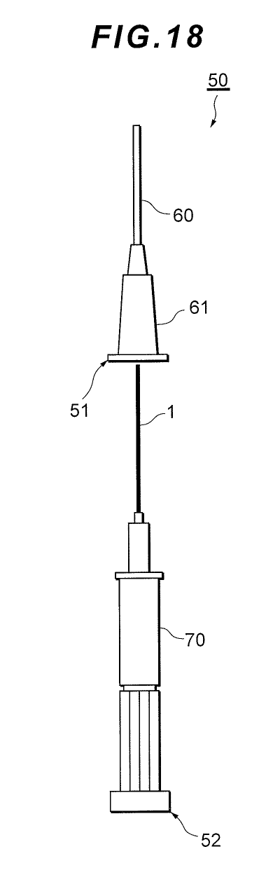

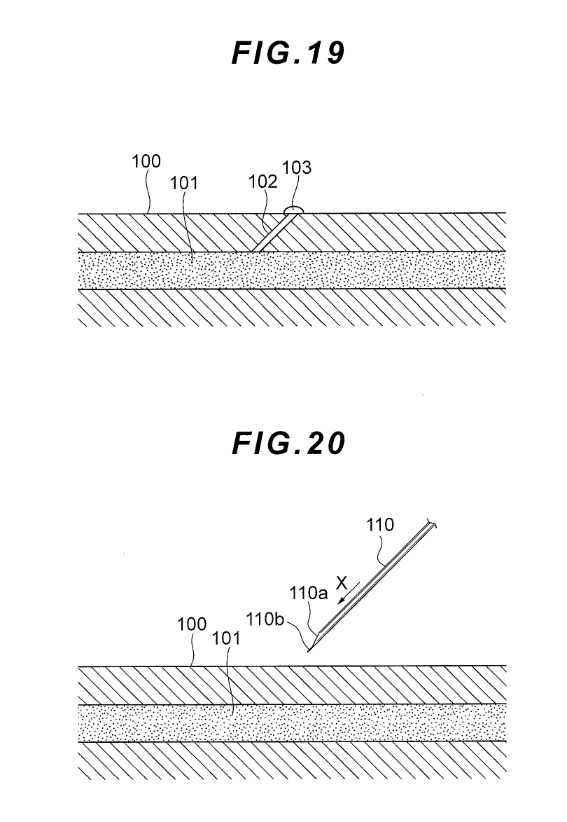

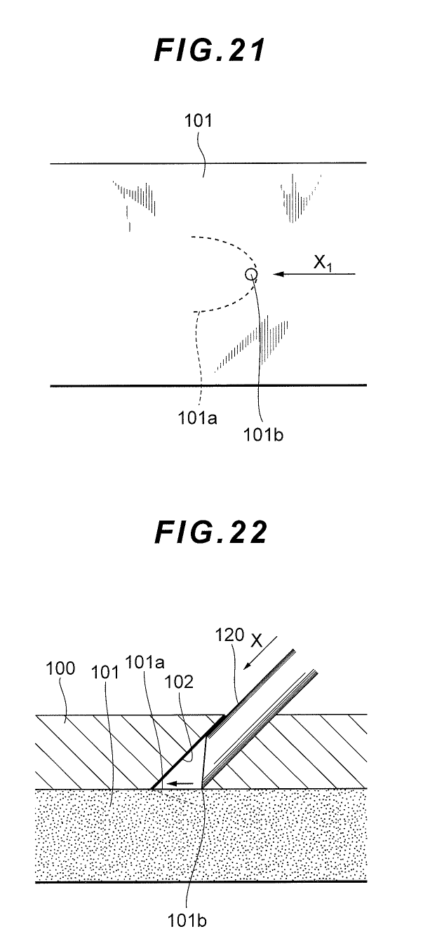

The present invention is also applicable to cases in which incision needle 1 is used as part of an indwelling needle. In this case, as shown in FIG. 18 for example, indwelling needle 50 has outer needle part 51 and inner needle part 52. Outer needle part 51 has outer needle 60 and connecting part 61 attached to the posterior end of this outer needle 60. Inner needle part 52 has incision needle 1 as the inner needle and holding part 70 or the like provided with a filter or the like, which is attached to the posterior end of incision needle 1. With incision needle 1 inserted into outer needle 60 with pointed extremity 21 protruding from the tip of outer needle 60, shunt blood vessel 101 is punctured with incision needle 1 with pointed extremity 21 uppermost as in the previous embodiment. Outer needle 60 is then inserted into shunt blood vessel 101, after which only incision needle 1 is removed from shunt blood vessel 101, leaving outer needle 60. A tube or the like is connected to connecting part 61 of outer needle 60, and blood is removed and returned through outer needle 60 to perform blood purification treatment or the like. Even if incision needle 1 is an indwelling needle, it may still be in solid cylindrical form.

### 2^{nd} Embodiment

In the 1^{st} embodiment above, the dull needle used as the puncture needle may be as follows.

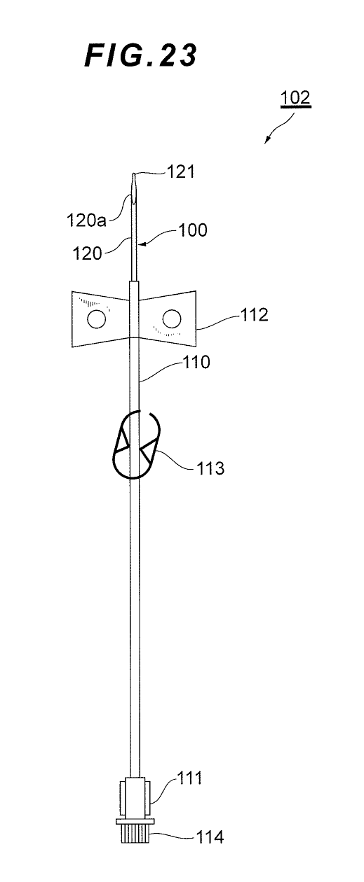

FIG. 23 is an explanatory drawing showing one example of a blood purification treatment instrument having dull needle 105 of this embodiment.

Blood purification treatment instrument 106 has dull needle 105 for puncturing the patient during blood purification treatment for example, tube 110 with dull needle 105 connected to the tip thereof, and connecting part 111 for connecting the posterior end of tube 110 to another tube. The part of tube 110 near dull needle 105 is provided with holding part 112, which is held by the operator when moving dull needle 105. Clamp 113 is attached to tube 110. Cap 114 fits onto connecting part 111.

Blood purification treatment instrument 106 is connected to another tube by connecting part 111 for example to thereby constitute part of a blood purification circuit having a hemopurification device (not shown). Blood purification treatment instrument 106 are attached to the end section on the blood removal side and the end section on the blood return side of the blood purification circuit, and remove and return blood via dull needles 105 during blood purification treatment. Blood purification treatment includes for example hemodialysis, plasma exchange, plasma adsorption, blood component elimination and the like.

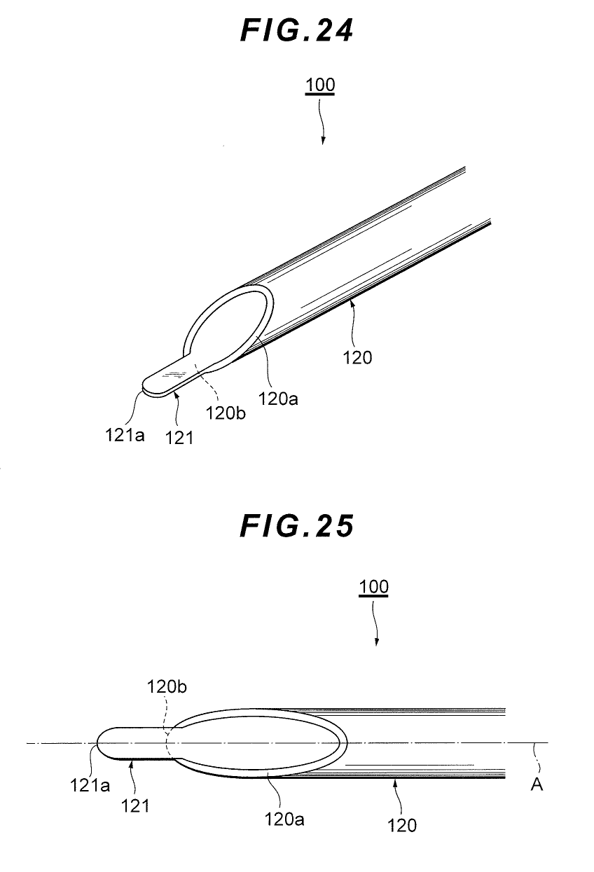

As shown in FIGS. 24, 25 and 26, dull needle 105 has tubular part 120 having angled end face 120a angled with respect to needle axis A at the tip of the needle, and protruding part 121 protruding forwards from tip 120b of angled end face 120a of tubular part 120. Protruding part 121 is formed with a narrower width (length in the right-left direction perpendicular to axis A) than tubular part 120. The thickness of protruding part 121 is the same as that of tubular part 120, and it is curved in an arc in the direction of width with the same thickness as tubular part 120 and the same curvature radius as that of the horizontal section (cross-section perpendicular to axial direction) of tubular part 120. The inner wall of protruding part 121 is continuous with the inner wall of tubular part 120, and the outer wall of protruding part 121 is continuous with the outer wall of tubular part 120.

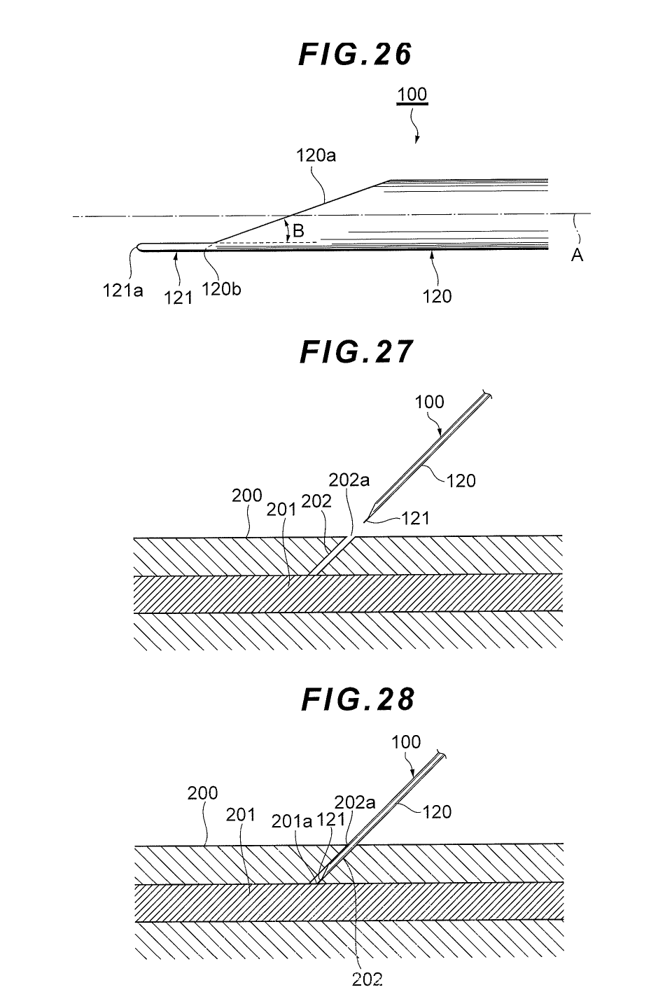

Tip 121a of protruding part 121 curves convexly towards the front. The curvature radius of tip 121a of protruding part 121 in the axial direction is set smaller than the curvature radius of tip 120b (shown by the dotted line in FIG. 25) of angled end face 120a for example. The radius of the curved part of protruding part 121 is set at 200 μm or more for example. The inclination angle B of angled end face 120a relative to the protruding direction of protruding part 121 (direction of needle axis A) as shown in FIG. 26 is set at less than 60°. This means that the angle formed by angled end face 120a with the edge of protruding part 121 connected to angled end face 120a (which is the same as inclination angle B) is less than 60°. The relationship of curvature radius Re of tip 121a of protruding part 121 and radius Ro of the outer diameter of tubular part 120 may be such as to fulfill 0.1×Ro≦Re<1.0×Ro. In FIG. 25, radius Ro is the distance between the outer surface and the center line (dotted line) of tubular part 120.

The outer diameter of tubular part 120 of dull needle 105 is preferably about 0.4 mm to 2.5 mm, and in this case the width of protruding part 121 in the direction of width is preferably about 0.02 mm to 2.50 mm. The length of protruding part 121 in the direction of needle axis A is preferably 2.5 mm to 5.00 mm. Dull needle 105 may be a stainless steel or other metal needle, or may also be a plastic needle. In the case of a plastic needle, it is desirable to use polytetrafluoroethylene, ABS resin (acrylonitrile-butadiene-styrene), polycarbonate, polypropylene, polyethylene or the like.

The operation of puncturing a shunt blood vessel using a dull needle 105 configured in this way is described next. The initial puncture has been accomplished with an ordinary needle prior to this operation, forming puncture route 202 from skin 200 to shunt blood vessel 201 in the patient's shunt area as shown in FIG. 31. Blood purification treatment instrument 106 having dull needle 105 has been removed from its sterile package and connected to a blood purification circuit.

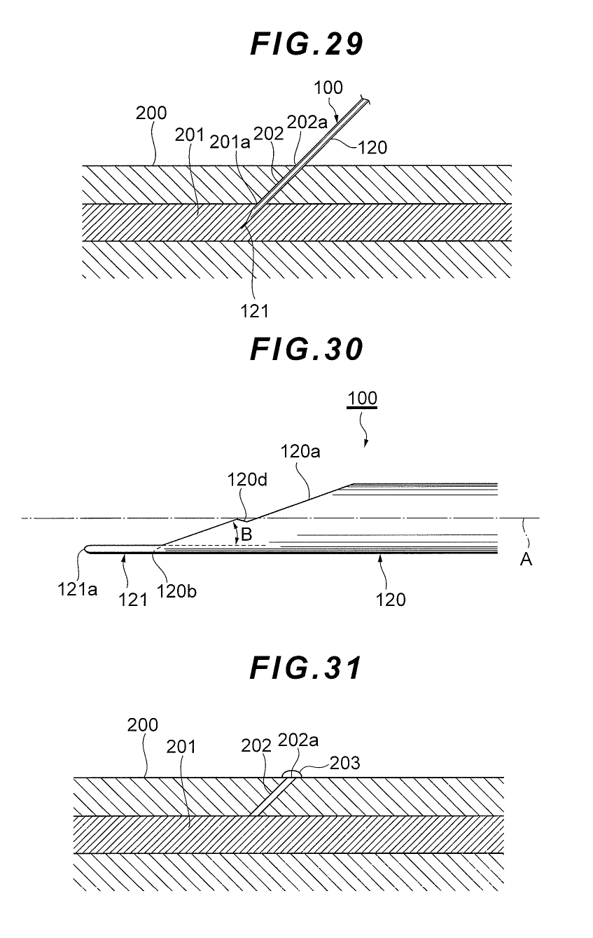

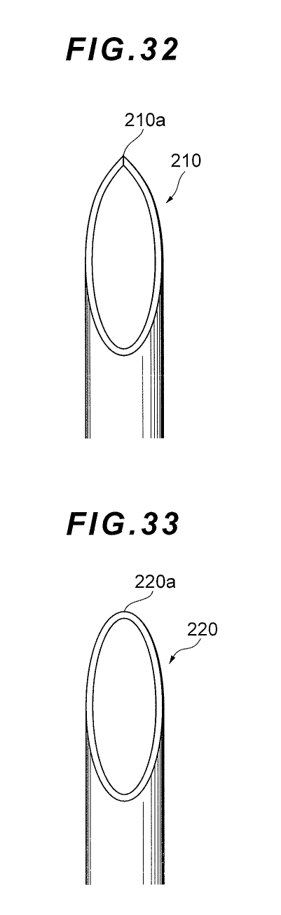

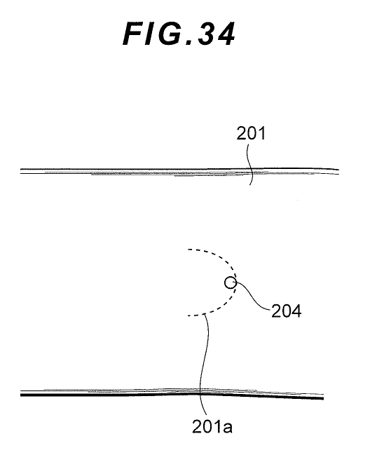

First, scab 203 on puncture route 202 is removed with dull needle 105 or the like as shown in FIG. 27, opening puncture hole 202a in skin 200. Dull needle 105 is then inserted into puncture route 202 via puncture hole 202a. After dull needle 105 is inserted, protruding part 121 reaches closed puncture hole 201a on the surface of shunt blood vessel 201 as shown in FIG. 28. Protruding part 121 of dull needle 105 is then pushed against puncture hole 201a as tip 121a of protruding part 121 searches for puncture start point 204 of U-shaped puncture hole 201a as shown in FIG. 34. When reverse U-shaped puncture hole 101a is formed using incision needle 1 as in the 1^{st} embodiment, the tip searches for puncture start point 101b of a reverse U-shaped puncture hole 101a such as that shown in FIG. 7. Once tip 121a of protruding part 121 is aligned with puncture start point 204 (101b), puncture hole 201a is opened starting from this puncture start point 204 as shown in FIG. 29, and dull needle 105 is inserted into shunt blood vessel 201. Blood is then removed and returned through dull needle 105, and blood purification treatment is performed using a blood purification circuit.

With the embodiment described above, because protruding part 121 is formed with a narrow width on tip 120b of angled end face 120a of dull needle 105, the sensation of shunt blood vessel 201 is transmitted more easily to the fingers from protruding part 121, thereby improving the ability of the needle to find contact start point 204. This facilitates and expedites the operation of puncturing shunt blood vessel 201. Moreover, because tip 121a of protruding part 121 is curves convexly towards the front, tip 121a of protruding part 121 is prevented from injuring or causing pain to the patient.

In this embodiment, moreover, the curvature radius of tip 121a of protruding part 121 in the axial direction is smaller than the curvature radius of tip 120b of angled end face 120a in the axial direction, so that the sensation of shunt blood vessel 201 is communicated more easily to the fingers via tip 121a of protruding part 121, thereby improving the ability of the needle to find contact start point 204.

Moreover, because the inclination angle B of angled end face 120a relative to the protruding direction of protruding part 121 is less than 60°, the resistance of dull needle 105 is less during puncture, and less force is required to puncture shunt blood vessel 201 with dull needle 105.

A preferred embodiment (2^{nd} embodiment) of the present invention was explained above with reference to the attached drawings, but the present invention is not limited by this example. Of course a person skilled in the art could conceive of various changes and modifications within the scope of the concepts described in the Claims, and these are naturally understood to fall within the technical scope of the present invention.

For example the shape of protruding part 121 of dull needle 105 is not limited to that described in the embodiment, and another shape may be used. For example, protruding part 121 may have a shape in which the width of tip 121a is narrower than the width of the other part. Also, in the embodiment the curvature radius of tip 121a of protruding part 121 in the axial direction is smaller than the curvature radius of tip 120b of angled end face 120a in the axial direction, but this relationship may be reversed. Angled end face 120a may also have a step 120d formed midway as shown in FIG. 30. When the inclination angle of angled end face 120a is different before and after step 120d, the angle with the largest inclination is called inclination angle B. Moreover, in the aforementioned embodiment dull needle 105 was the needle used for blood purification treatment, but it may also be one used in the so-called buttonhole puncture method. For example, the present invention is applicable to dull needles used to administer drugs by syringe or to take blood sample.

### 3^{rd} Embodiment

In either one or both of the 1^{st} embodiment and 2^{nd} embodiment described above, a needle cap may be mounted on the needle, and a scab at the entrance of the puncture route can be removed with this needle cap. This example is explained below as the 3^{rd} embodiment.

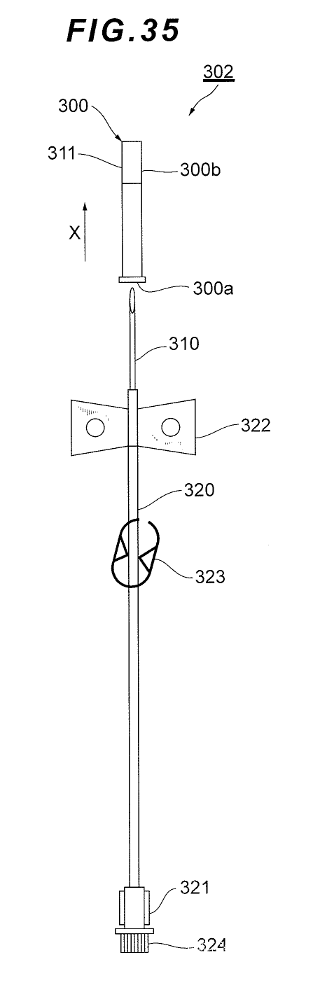

FIG. 35 is an explanatory drawing showing one example of blood purification treatment instrument 302 having needle cap 300 of this embodiment.

Needle cap 300 is formed in a roughly cylindrical shape for example to allow insertion of needle 310. Needle cap 300 has holding part 301 for holding needle 310. Needle cap 300 has opening 300a formed at the posterior end in the direction of length (direction X of insertion axis of needle 310), and needle 310 is inserted through this opening 300a. Scab removal part 311 is formed at tip 300b of needle cap 300.

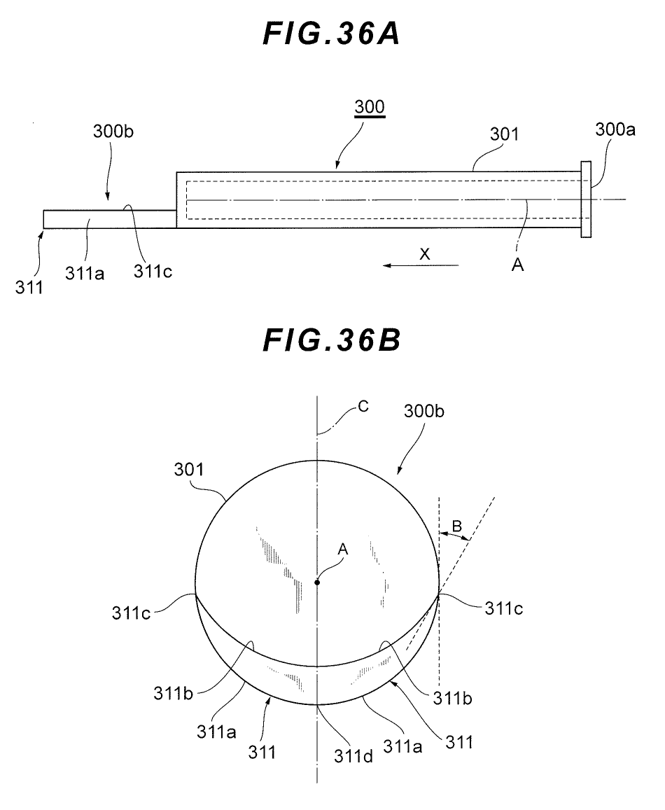

As shown in FIGS. 36A and 36B for example, scab removal part 311 comprises outer surface 311a in the form of a circular arc curving around insertion axis A of needle 310, and inner surface 311b located inside the outer surface, and is formed substantially in a crescent shape in cross-section for example. Sharp top edge 311c with a width in insertion axis direction X of needle 310 is formed by outer surface 311a and inner surface 311b. If sharp angle B of outer edge 311c is small, the edge is sharp and easy to insert into space between the scab and the skin, but strength is reduced, while if the angle is large, the edge is stronger but harder to insert, so the angle is preferably 5° to 89° or more preferably 5° to 35°.

Central line C passing between the left and right passes through base 311d of tip 300b for example, and outer surface 311a is formed starting from base 311d to a distance of about 90° away from base 311d for example. The radius of outer surface 311a is preferably about 0.5 to 5.2 mm for example. The length of the arc of outer surface 311a is preferably 5 to 50% of the maximum outer diameter of holding part 301. Inner surface 311b is formed with a convex curve towards outer surface 311a. Outer surface 311a and inner surface 311b meet at a sharp angle to form outer edge 311c. Outer edge 311c is formed parallel to insertion axis direction X of needle 310, and is about 0.5 to 5.2 mm wide for example.

Scab removal parts 311 are formed symmetrically on both sides of central line C. Scab removal parts 311 are formed so that outer edges 311c are reverse to each other on the circumference of tip 300b. That is, in FIG. 36B the top edges face upward, with left outer edge 311c on the side that curves rightward and right outer edge 311c on the side that curves leftward. The left and right scab removal parts 311 form scab removal part 311 as a whole in an upward-curving crescent shape. That is, scab removal part 311 as a whole comprises of outer surface 311a in the form of a roughly semicircular arc with inner surface 311b inside the arc and outer edges 311c on both sides.

Although outer edges 311c of scab removal part 311 need to be sharp enough to detach the scab, the sharpness needs to be controlled so that the skin will not be damaged during scab removal and so that the sterile package will not be damaged during transport.

The material of needle cap 300 is not particularly limited, and a resin or metal material may be used for example. For example, needle cap 300 can be molded from a synthetic resin material, in which case it can be designed with a suitable strength, and can be manufactured cheaply in a complex shape. It can also be incinerated as medical waste. A material that can be injection molded and can withstand sterilization is preferred as a synthetic resin material. Sterilization means EOG sterilization, autoclave sterilization, gamma ray sterilization, electron beam sterilization or the like, and the material may be able to withstand at least one of these. Specifically, desirable examples include polypropylene, polyethylene, polytetrafluoroethylene, ABS resin (acrylonitrile-butadiene-styrene), polycarbonate and the like. The dimensions of needle cap 300 differ depending on the outer diameter of the needle and the hub diameter, but a bore/outer diameter ratio of between 1 mm/2 mm and 5 mm/8 mm and a length of about 20 mm to 60 mm are preferred.

Needle cap 300 is used with blood purification treatment instrument 302 as shown in FIG. 35 for example. Blood purification treatment instrument 302 comprises needle 310 for puncturing a patient during blood purification treatment for example, tube 320 with needle 310 connected to the tip thereof, and connecting part 321 for connected the posterior end of tube 320 to another tube. The part of tube 320 near needle 310 is provided with holding part 322, which the operator holds when moving needle 310. Clamp 323 is also attached to tube 320. Cap 324 fits onto connecting part 321. Needle 310 may be a very sharp ordinary needle for forming a puncture route, or a less sharp dull needle for inserting into a puncture route. Needle 310 may be made of stainless steel or another metal, or it may be a plastic needle.

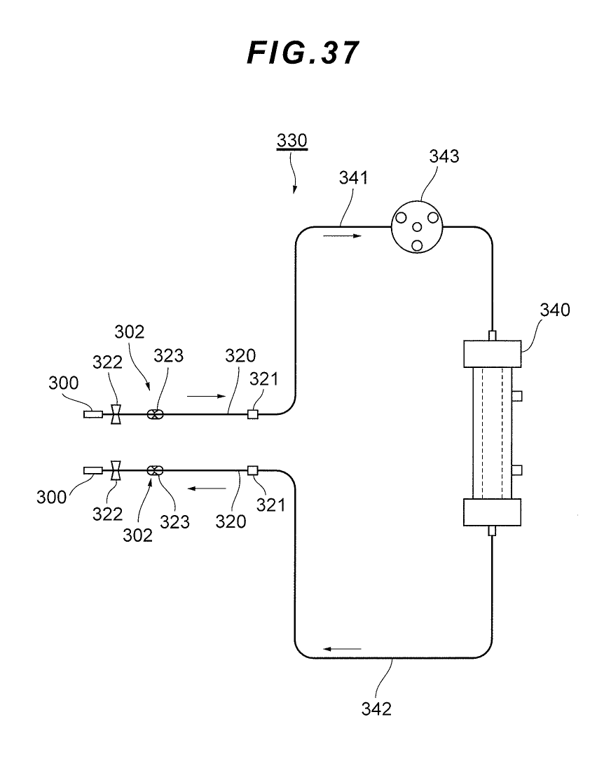

Blood purification treatment instrument 302 can be applied to blood purification circuit 330 as shown in FIG. 37 for example. Blood purification circuit 330 comprises hemopurification device 340, which may be a hollow-fiber module that purifies blood by separating or adsorbing blood components for example, blood supply circuit 341, which supplies removed blood to hemopurification device 340, and blood return circuit 342, which returns blood that has passed through hemopurification device 340. Blood supply circuit 341 is provided with pump 343. Blood purification treatment instruments 302 are connected to the end section on the blood removal side of blood supply circuit 341 and the end section on the blood return side of blood return circuit 342 for example, and constitute part of blood purification circuit 330. Blood purification circuit 330 can be used for blood purification treatment such as hemodialysis, plasma exchange, plasma adsorption, blood component elimination or the like for example.

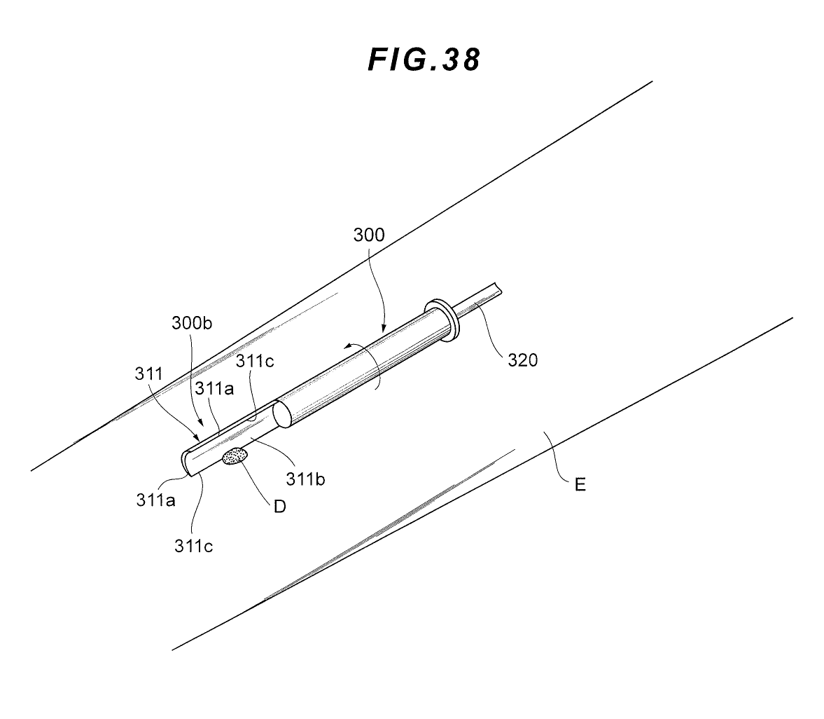

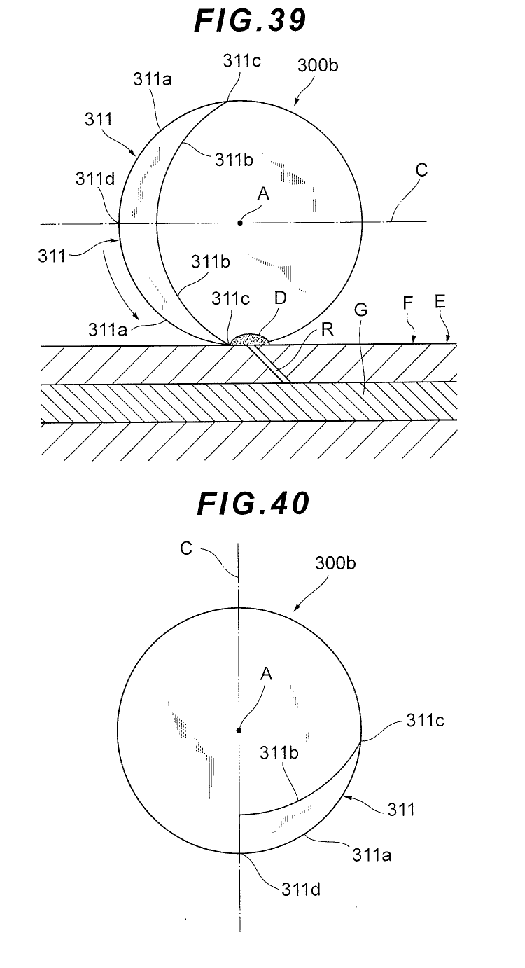

When performing blood purification treatment using needle caps 300 and blood purification treatment instruments 302 configured as described above, for example, blood purification treatment instruments 302 are removed from their sterile packages and connected to both ends of blood purification circuit 330 as shown in FIG. 37. Next, scabs are removed from the patient using needle caps 300 of blood purification treatment instruments 302 for example. To do this, the operator arranges scab removal part 311 of needle cap 300 near scab D of forearm E as shown in FIGS. 38 and 39 for example, and then inserts outer edge 311c of scab removal part 311 into the space between scab D and skin F. Scab removal part 311 is then rotated around insertion axis A of needle 310 to rotate outer edge 311c and detach scab D. Needle cap 300 is then removed, needle 310 is inserted into puncture route R with the scab removed, and shunt blood vessel G under skin F is punctured with needle 310. Blood is then removed and returned through needles 310, and blood purification is performed using blood purification circuit 330.

In the embodiment described above, because sharp top edge 311c with a width in the direction of insertion axis X of needle 310 is formed on tip 300b of needle cap 300, scab D can be detached by rotating outer edge 311c around insertion axis A of needle 310. This makes the area of contact between outer edge 311c and scab D easier to see because the visual field of the operator is no longer obstructed by the needle cap or the operator's own fingers when removing scab D. The operation of aligning outer edge 311c with scab D is also facilitated by the width of top edge 311c used to detach scab D. This makes the operation of removing the scab easier, so that it can be removed without damage to the skin.

In the embodiment described above, scab removal part 311 is formed on both left and right sides of the center line C of tip 300b of needle cap 300, and each top edge 311c is arranged to be reverse to each other on the circumference of tip 300b. The operation of detaching scab D can thus be performed from either direction, allowing scab D to be removed from the side with greater visibility.

Because inner surface 311b of scab removal part 311 curves convexly towards outer surface 311a, the angle of outer edge 311c can be made sharper, facilitating removal of scab D.

The aforementioned needle cap 300, which is used with needles including dull needles and incision needles for puncturing during blood purification treatment, can help ensure more rapid and efficient operations and reduce the burden on the patient because it allows a scab D to be removed easily during blood purification treatment, which must be performed multiple times on the same patient.

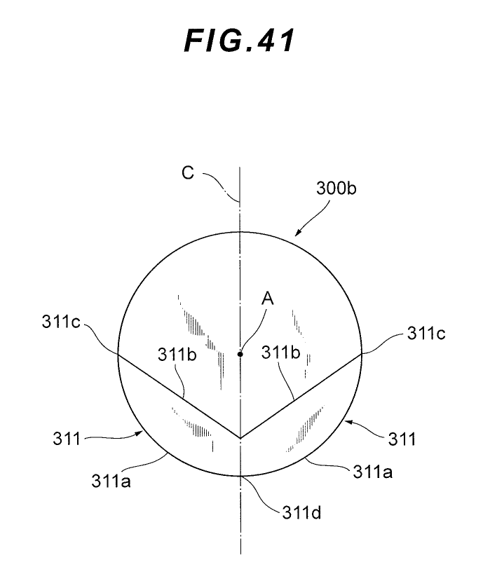

In the embodiment described above, scab removal part 311 was provided on both left and right sides of central line C, but it may also be provided on only one side as shown in FIG. 40 for example. Inner surface 311b of the scab removal part 311 described above curves convexly towards outer surface 311a, but it may also be a flat inclined surface as shown in FIG. 41.

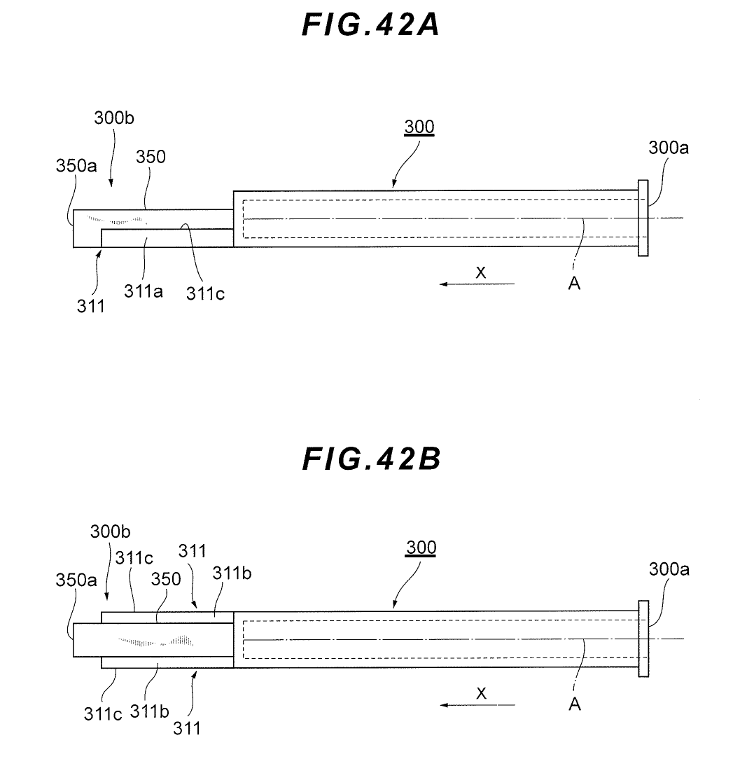

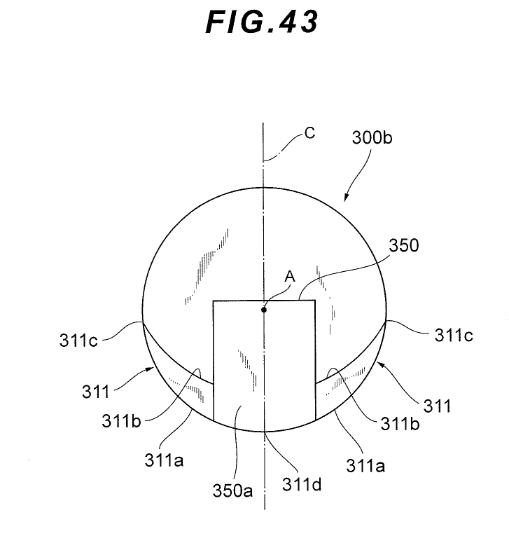

In the needle cap 300 described in the embodiment above, a protruding part may also be formed on tip 300b. This case is explained below. For example, as shown in FIGS. 42A, 42B and 43, protruding part 350 with flat anterior surface 350a is formed on tip 300b of needle cap 300, extending further forward than outer edge 311c. Protruding part 350 is formed for example as a long rectangular column extending in direction X of the insertion axis of needle 310, and is formed on center line C extending forwards from the base of tip 300b. Protruding part 350 is formed with a width of 0.2 to 4.0 mm and a height of 0.2 to 5.2 mm for example. Outer surface 311a of scab removal part 311 extends outwards around the circumference of tip 300b from protruding part 350, and outer edge 311c is formed at the periphery of this outer surface 311a away from the protruding part. Inner surface 311b is formed from outer edge 311c as far as the side of protruding part 350. Scab removal part 311 is formed on both the left and right sides of protruding part 350. In this example, other components are the same as in the previous embodiments, and are indicated with the same symbols, without further explanation.

In this example, because protruding part 350 is formed on tip 300b of needle cap 300, the tip of needle cap 300 is prevented from opening a hole in the sterile package during transport for example. Needle 310 can therefore be transported more securely.

Moreover, tip 300b can be made stronger because protruding part 350 is formed on the left right sides of the center line C of tip 300b of needle cap 300, which passes between the left and right. This allows the thickness of scab removal part 311 to be reduced for example, so that the angle of outer edge 311c can be sharper. This facilitates the operation of removing scab D.

Because scab removal part 311 is formed on both the left and right sides of protruding part 350, moreover, scab D can be removed from either the left or right side, allowing the scab to be removed from the side with greatest visibility.

Scab removal part 311 can also be provided on only one side of protruding part 350 in this example. Protruding part 350 may also have a solid cylindrical or other shape for example. Moreover, the strength of tip 300b of needle cap 300 differs according to the material properties and the structure of the molded body, but it preferably has the property of not penetrating under loads of 3000 mN or less in an evaluation test using silicon rubber. Using a load measuring device (Model 1605N; Aikoh Engineering), this evaluation test was performed by puncturing silicon rubber (SR-50; Tigers Polymer) of thickness 1.05 mm, diameter 40 mm Φ with a 17G needle, and then pressing with tip 300b of needle cap 300, evaluating penetration, and measuring the load during penetration. In this test, a conventional needle cap penetrated under a load of less than 2700 mN, while needle cap 300 with protruding part 350 of this embodiment did not penetrate under loads of 3000 mN or less. The details are explained in the examples.

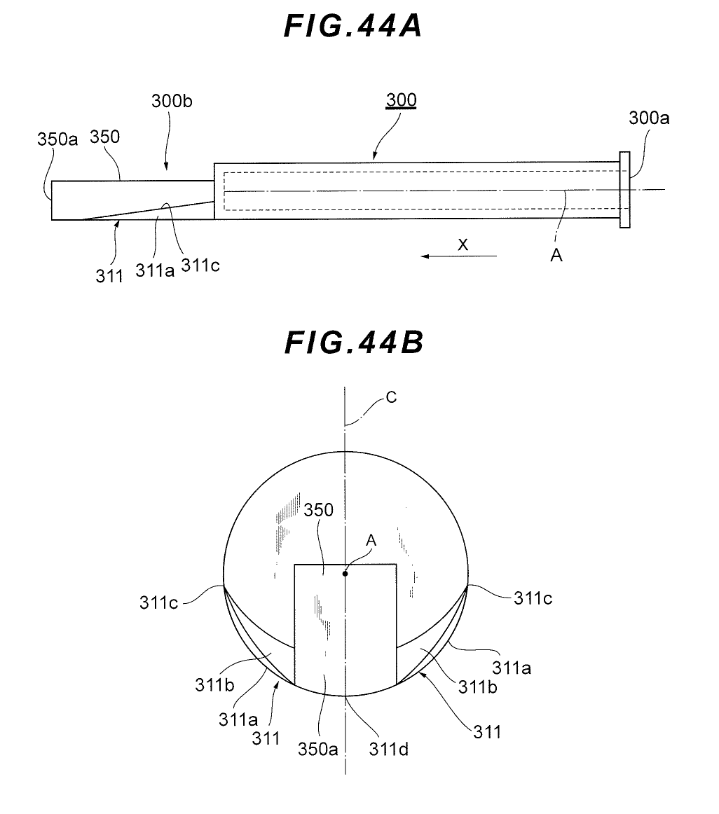

In the embodiment above, as shown in FIGS. 44A and 44B for example, inner surface 311b may be angled in direction X of the insertion axis of needle 310, so that scab removal part 311 tapers gradually towards the front. In this case, scab removal part 311 has a thick part and a thin part, and scab D can be removed with whichever part is easier to use according to the shape and size of scab D. This example can also be applied to a needle cap 300 having no protruding part 350.

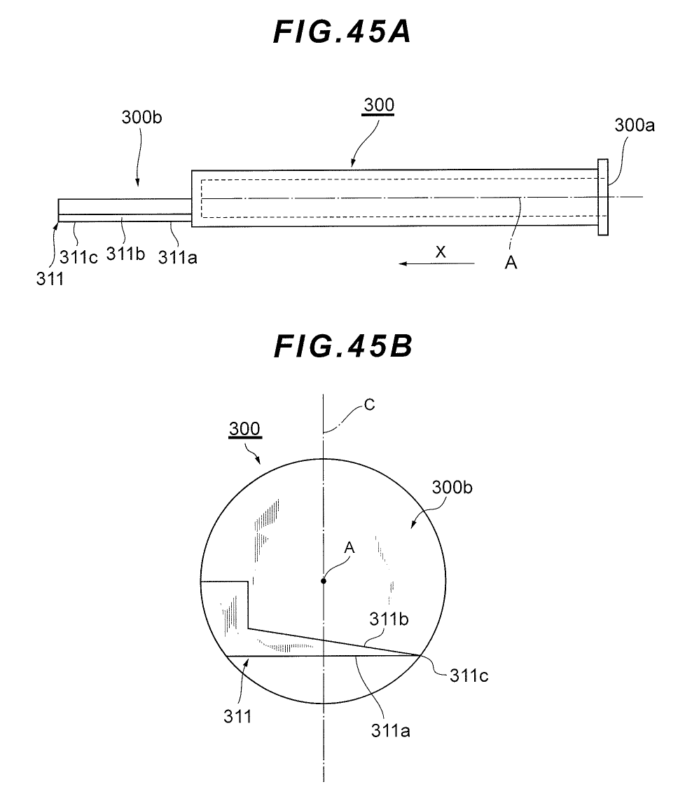

Instead of the curved outer surface 311a in the embodiment above, as shown in FIGS. 45A and 45B for example, scab removal part 311 may have flat outer face 311a formed in needle insertion axis direction X. Other components are the same as in the embodiment described above, and sharp top edge 311c with a width in insertion axis direction X of needle 310 is formed by outer face 311a and inner surface 311b. In this example as well, because scab D can be detached by rotating outer edge 311c around insertion axis A of needle 310, the operator's field of vision is not obstructed by the needle cap or by the operator's own fingers, and the area of contact between outer edge 311c and scab D is easy to see. Since outer edge 311c for detaching scab D has a width, moreover, it is easy to align outer edge 311c with scab D. In this example, the operation of scab removal can be easily performed without damage to the skin.

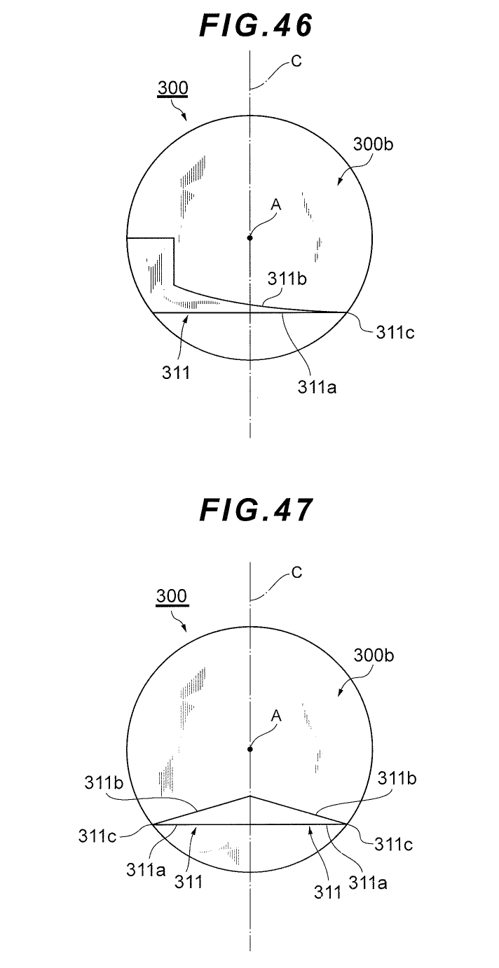

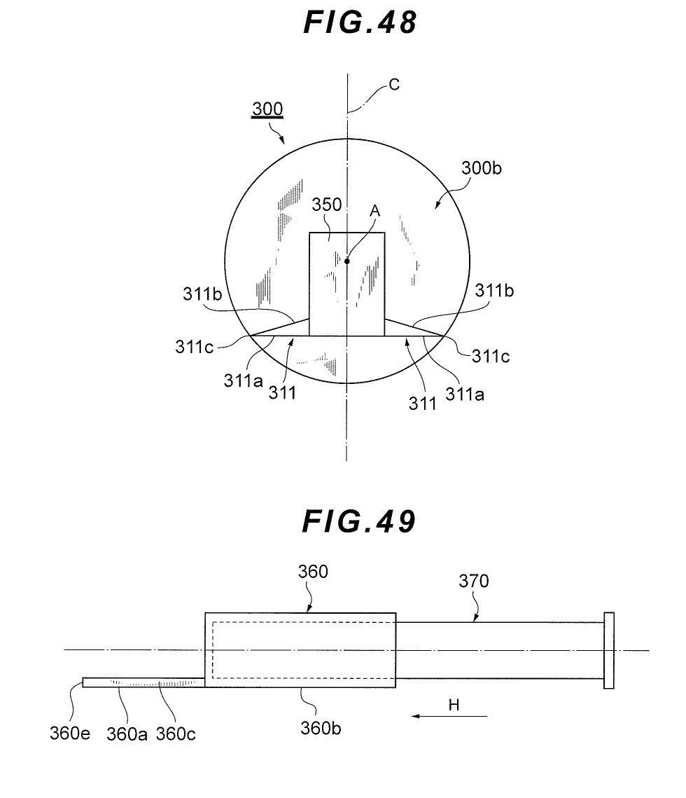

Inner surface 311b was flat in this example, but as shown in FIG. 46, inner surface 311b may also be curved convexly towards outer surface 311a as described in the aforementioned embodiment. Moreover, scab removal parts 311 can also be formed on both sides of right-left center line C as shown in FIG. 47, and outer edges 311c on both sides can be formed reverse to each other on the right and left sides of tip 300b. Moreover, as described in the embodiment above, tip 300b can be formed with protruding part 350 as shown in FIG. 48. Outer face 311a is formed on both left and right sides of protruding part 350, and outer edges 311c are formed at the periphery of this outer face furthest from protruding part 350. Outer face 311a may also be formed on only one side of protruding part 350.

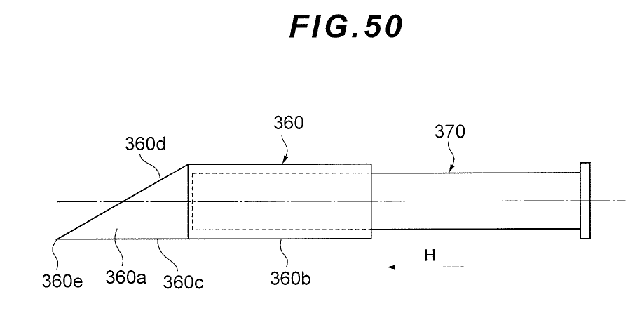

From a different perspective, the present invention may also be a scab removal tool for buttonhole puncture, attached to a needle cap. As shown in FIGS. 49 and 50, scab removal tool 360 has triangular plate-shaped tip 360a, which is attached to cylindrical or square columnar base 360b. Tip 360a has one side face 360c parallel to the long axial direction H of cylindrical needle cap 370, and another diagonal side face 360d, and sharp tip 360e is formed by these side faces 360c and 360d. Base 360b is connected to needle cap 370. In this example, the operation of scab removal can be easily performed by tip 360c, without damage to the skin.

Scab removal tool 360 may be formed as a unit with needle cap 370, or needle cap 370 may fit into it as a separate unit. Scab removal tool 360 may also be fixed to a sterile package containing a puncture needle. In this case, the sharp tip of scab removal tool 360 is prevented from rupturing the sterile package during transport.

A preferred embodiment (3^{rd} embodiment) of the present invention was explained above with reference to the attached drawings, but the present invention is not limited to this example. Of course a person skilled in the art could conceive of various changes and modifications within the scope of the concepts described in the Claims, and these are naturally understood to fall within the technical scope of the present invention.

For example, in the embodiment described above, needle cap 300 is used with needle 310 for puncturing during blood purification treatment, but the present invention can also be used with needles for puncturing when blood sample is taken or drug is administered with a syringe or the like.

### EXAMPLES

### Examples Corresponding to 1^{st} Embodiment

The results of performance evaluation testing of incision needle 1 described in the 1^{st} embodiment are given below. Performance evaluation testing 1 and performance evaluation testing 2 are tests for evaluating the puncturing performance of incision needles.

### (Evaluation Test 1)

In evaluation test 1, the puncture needles of each sample were pushed through silicon rubber (SR-50; Tigers Polymer) of thickness 1.05 mm, diameter 40 mm Φ with a load measuring device (Model 1605N; Aikoh Engineering) at an angle of 30° with the angled end face facing down (pointed extremity on top), and the necessary load was measured. The test conditions were room temperature 26° C., penetration speed 50 mm/min. The results of this evaluation test are shown in Table 1.

The sample of Example 1 was a incision needle (type shown in FIGS. 2 to 4) formed as described in the aforementioned embodiment with reverse cutting edges 31 on pointed extremity 21, while the sample of Example 2 was a incision needle (type shown in FIGS. 10 to 12) formed as described in the aforementioned embodiment with reverse cutting edges 31 behind pointed extremity 21. The sample of Example 3 is a solid cylindrical incision needle (type shown in FIGS. 14 to 15) formed with reverse cutting faces 31, and having pointed extremity 21 bent towards the central axis. The comparative example is a conventional needle in which the pointed extremity of the angled end face is not bent. The inclination angle of the angled end face of this conventional needle relative to the central axis is 15°.

TABLE 1
Puncture test

| Sample | Load at puncture (mN) |
|---|---|
| Example 1 (type of FIGS. 2 to 4) | 2089 ± 108 |
| Example 2 (type of FIGS. 10 to 12) | 1954 ± 12 |
| Example 3 (type of FIGS. 14 to 15) | 2612 ± 215 |
| Comparative Ex. | Failed to puncture |

While the incision needles of Examples 1, 2 and 3 punctured at loads of 2089 mN, 2247 mN and 2612 mN, respectively, the needle of the Comparative Example did not puncture the silicon rubber even at a load of 3300 mN.

### (Evaluation Test 2)

A cellulose sheet (about 150 μm) was affixed to the nylon nonwoven surface of a nylon nonwoven/urethane laminate (Scotch: 74×115×30 mm thick) to prepare a model shunt blood vessel. Puncture was performed on the surface of the cellulose sheet with a incision needle of the invention (type of FIGS. 2 to 4 with back-cut tip) as Example 1 and a incision needle of the invention (side-cut type of FIGS. 10 to 12) as Example 2 at an angle of about 30° with the angled end face facing down. Similarly, a conventional 17G puncture needle (Terumo) was also tested by conventional methods at an angle of about 30° with the angled end face facing up. In addition, a 17G dull needle (Nipro) was pushed forward from the front side of the puncture mark so as to graze the surface of the cellulose, and the puncture condition was evaluated.

TABLE 2
Puncture evaluation

| Sample | Puncture condition |
|---|---|
| Example 1 | 3 |
| Example 2 | 3 |
| Comparative Ex. | 1 |

Puncture condition evaluation:
1 (poor), 2 (normal), 3 (good)

The puncture condition was good with the incision needles of Examples 1 and 2, while puncture failed with the conventional needle of the Comparative Example.

### Examples Corresponding to 2^{nd} Embodiment

The results of a performance evaluation test of the dull needle 105 described in this embodiment are shown below. Evaluation test 1 is a test to evaluate puncture performance using dull needle 105. Evaluation test 2 is a test to evaluate puncture resistance using dull needle 105.

### (Evaluation Test 1)

The subjects being punctured in evaluation test 1 were 20 patients who were on hemodialysis more than 3 years and were receiving hemodialysis 3 times weekly. On the second hemodialysis session in the week in each patient, shunt blood vessel was punctured with either the dull needle 105 of the present invention shown in FIG. 24 or the conventional dull needle 220 shown in FIG. 33. The time from insertion of the dull needle into puncture route 202 to puncturing of shunt blood vessel 201 by the dull needle was measured. These puncture times are shown in Table 1 below.

TABLE 1
Puncture performance evaluation test

| Sample | Puncture time (seconds) |
|---|---|
| Dull needle of invention (shown in FIG. 24) | 2.6 ± 1.5 |
| Conventional dull needle (shown in FIG. 33) | 4.2 ± 2.8 |

Dull needle 105 of the present invention had a shorter puncture time than conventional dull needle 220.

### (Evaluation Test 2)

In evaluation test 2, after silicon rubber (SR-50; Tigers Polymer) of thickness 1.05 mm, diameter 40 mm was punctured with a conventional 17G needle, each, dull needle was passed through the puncture route of the conventional 17G needle in the rubber, and the necessary load in each case was measured using a load measuring device (Model 1605N; Aikoh Engineering). The test conditions were room temperature 26° C., penetration speed 50 mm/min. The results of this evaluation test 2 are shown in Table 2. The maximum required load in Table 2 is the maximum load required to achieve penetration with each sample, while the necessary contact point load was the load required for the contact point between protruding part 121 and angled end face 120a to pass through the silicon rubber.

The Samples of Examples 1, 2, 3 and 4 and Comparative Example 1 are dull needles having contact point angles (inclination angles) B between protruding part 121 and angled end face 120a of 12.0°, 11.7°, 25.1°, 28.4° and 65.0°, respectively. The sample of Comparative Example 2 was the dull needle shown in FIG. 33, which has no protruding part 121.

TABLE 2
Penetration resistance test

| Sample name | Maximum necessary load | Necessary contact point load |
|---|---|---|
| Example 1 | 596.8 ± 31.8 mN | 26.1 ± 22.4 mN |
| Example 2 | 651.4 ± 38.6 mN | 11.4 ± 26.2 mN |
| Example 3 | 744.0 ± 35.0 mN | 0.0 ± 0.0 mN |
| Example 4 | 699.0 ± 49.0 mN | 35.0 ± 15.0 mN |
| Comparative Ex. 1 | 1265.8 ± 161.2 mN | 167.8 ± 21.7 mN |
| Comparative Ex. 2 | 1181.0 ± 40.3 mN | — |

The maximum necessary load in Examples 1 to 4 having a contact angle B of less than 60° was much smaller than in Comparative Examples 1 and 2. The necessary contact point load in Examples 1 to 4 was also less than 100 mN, which was much smaller than in Comparative Example 1. This shows that penetration resistance is greatly reduced and puncture can be accomplished more smoothly if contact angle B is less than 60°.

### Examples Corresponding to 3^{rd} Embodiment

The results of performance evaluation testing of a needle cap having a tip with the specific shape indicated in this embodiment are given below. Evaluation test 1 is a test to evaluate the penetration performance of the tip of the needle cap, while evaluation test 2 is a test to evaluate the scab removal performance.

### (Evaluation Test 1)

Evaluation test 1 was performed using a load measuring device (Model 1605N; Aikoh Engineering). Precisely, silicon rubber (SR-50; Tigers Polymer) of thickness 1.05 mm, diameter 40 mm Φ was punctured with a 17G needle, the punctured point was pressed with tip 300b of needle cap 300 of each sample, it was examined whether or not tip 300b penetrated the silicon rubber at the point, and the load was measured during penetration. The test conditions were room temperature 26° C., penetration speed 50 mm/min. The results of this evaluation test 1 are shown in Table 1.

The sample of Example 1 was needle cap 300 having a protruding part 350 such as that shown in FIG. 44, in which inner surface 311b and outer edge 311c are tapered towards the front. The sample of Example 2 was needle cap 300 having a protruding part 350 such as that shown in FIG. 42, in which inner surface 311b and outer edge 311c are horizontal rather than tapered. The sample of Example 3 is needle cap 300 having a protruding part 350 similar to that of Example 2, but with inner surface 311b being an angled plane surface as shown in FIG. 41 rather than a curved surface. The sample of Comparative Example 1 is a needle cap with a point extending towards the front as described in Japanese Translation of PCT Application No. 2009-1542343.

TABLE 1
Penetration test

| Sample | Load at puncture (mN) |
|---|---|
| Example 1 | Failed to penetrate |
| Example 2 | Failed to penetrate |
| Example 3 | Failed to penetrate |
| Comparative Ex. 1 | 2543 ± 142 |

While the sample of Comparative Example 1 penetrated the silicon rubber under a load of 2543 mN, the samples of Examples 1 to 3 failed to penetrate the silicon rubber even under a load of 3000 mN.

### (Evaluation Test 2)

In evaluation test 2, scab detachment was performed using the needle cap of each sample, the detachment status was evaluated, and the scab removal time was measured. Evaluation test 2 was performed on a shunt site of the upper arm with a scab formed by previous puncture on the second dialysis day, with the subjects being 4 end stage renal failure hemodialysis patients who had started dialysis at least 3 years before and were currently undergoing hemodialysis 3 times a week. The results of this evaluation test 2 are shown in Table 2. The samples of Examples 1 to 3 and Comparative Example 1 were the same as the samples used in evaluation test 1 above. The sample of Comparative Example 2 was a 17G injection needle (Terumo). The sample of Comparative Example 3 was a 17G AVF dull needle (Nipro). The reason why an injection needle and AVF dull needle were used for scab removal as Comparative Examples 2 and 3 is that needles are often used to remove scabs in clinical practice.

The scab detachment status was evaluated in 4 levels, while for the scab removal time, the total scab removal time from the initial operation of removing the blood purification treatment instrument including each sample from its sterile package to the beginning of puncture with the needle following removal of the scab with the sample was measured, as well as the scab removal time, which was the time spend actually removing the scab.

TABLE 2
Scab detachment evaluation

| | | Total scab | Scab removal |
|---|---|---|---|
| Sample | Detachment | removal time (sec) | time (sec) |
| Example 1 | 4 | 15 | 3 |
| Example 2 | 4 | 17 | 4 |
| Example 3 | 4 | 16 | 4 |
| Comparative Ex. 1 | 1 | 22 | 9 |
| Comparative Ex. 2 | 2 | 32 | 7 |
| Comparative Ex. 3 | 2 | 24 | 7 |

Detachment status:
1 (poor), 2 (normal), 3 (good), 4 (very good)

In Comparative Example 1, the detachment status was poor and the scab removal time was long because the site was hard to see and detachment involved sliding the skin. The total scab removal time was also long because the cap was sharp and required careful operation.

The detachment status was normal as indicated by “2” in the table in Comparative Examples 2 and 3. In Comparative Example 2, the total scab removal time was long because the operation of removing another AVF dull needle from its sterile package intervened after scab removal. In Comparative Example 3, the needle had to be disinfected with an alcohol wipe to remove the adhering scab and sterilize the needle after scab removal, and this disinfection with the alcohol wipe made the total scab removal time longer. Great precision was required in Comparative Examples 2 and 3 because of the danger of skin damage from the needle tip, but this did not affect the scab removal time.

The detachment status was 4 (very good) in the examples, the total scab removal times and scab removal times were short, and confirmation with the naked eye was possible, allowing for a safe detachment operation.

I (We) claim:

1. A method of puncturing a shunt blood vessel, comprising a step of puncturing a shunt blood vessel with a tubular or cylindrical incision needle and thereby forming an incision on a surface of the shunt blood vessel, wherein
the incision needle has an angled end face at the tip of the needle, and the tip of the angled end face has a pointed extremity that protrudes apically from a hypothetical angled plane formed by cutting straight through the tip of the needle at an angle, and
the step of forming the incision comprises:
a step of arranging the incision needle so that the angled end face faces the skin;
a step of puncturing the skin with the pointed extremity of the incision needle and inserting the needle toward the shunt blood vessel to thereby form a puncture route; and
a step of inserting the pointed extremity of the incision needle into the shunt blood vessel to thereby form a reverse U-shaped incision that opens towards the front side, with the apex of the incision being on the far side in the direction of insertion.

2. The puncture method according to claim 1, wherein the incision needle is a needle in which the pointed extremity is bent towards the central axis of the needle.

3. The puncture method according to claim 1, wherein the incision needle is a needle in which a cutting face is formed on at least a part, in the angled end face, that includes the pointed extremity.

4. The puncture method according to claim 3, wherein the incision needle has the cutting face extending from the pointed extremity to the angled surface of the angled end face.

5. The puncture method according to claim 1, wherein the incision needle has a reverse cutting face on the reverse surface of the angled end face.

6. The puncture method according to claim 5, wherein the incision needle has the reverse cutting face on at least the pointed extremity.

7. The puncture method according to claim 5, wherein the incision needle has the reverse cutting face on both sides of the reverse surface of the angled end face formed posterior to the pointed extremity.

8. The puncture method according to claim 1, wherein the incision needle has another pointed extremity pointing apically behind the pointed extremity of the angled end face.

9. The puncture method according to claim 1, wherein the incision needle has a groove, on the reverse surface, for accepting blood of the shunt blood vessel from the tip of the angled end face.

10. The puncture method according to claim 1, further comprising a step of penetrating the puncture route in the skin with a puncture needle and inserting the puncture needle into the shunt blood vessel through the incision, after formation of the reverse U-shaped incision.

11. The puncture method according to claim 10, wherein
the puncture needle has a tubular part with an angled end face angled with respect to the needle axis and a protruding part that protrudes forwards from the tip of the angled end face of the tubular part, and wherein
the tip of the protruding part is narrower than the tubular part, and is curved convexly towards the front.

12. The puncture method according to claim 11, wherein the curvature radius of the tip of the protruding part of the puncture needle is smaller than the curvature radius of the tip of the angled end face.

13. The puncture method according to claim 11, wherein the inclination angle of the angled end face relative to the protruding direction of the protruding part of the puncture needle is less than 60°.

14. The puncture method according to claim 10, further comprising a step of removing a scab formed at the entrance of the puncture route on the skin surface with a needle cap of the puncture needle before inserting the puncture needle into the puncture route.

15. The puncture method according to claim 14, wherein
the needle cap has a hollow holding part for holding the needle and a scab removal part at the tip of the holding part, and
the scab removal part is formed from a circular arc-shaped outer surface that curves around the needle insertion axis and an inner surface located inside the outer surface, and in the scab removal part a sharp top edge with a width in the direction of the needle insertion axis is formed by the outer surface and inner surface.

16. The puncture method according to claim 15, wherein
the scab removal part is formed substantially in a crescent shape in cross-section, and
the angle formed by the arc of the outer surface and the arc of the inner surface at the top edge is 5° to 89° in this crescent shaped cross-section.

17. The puncture method according to claim 15, wherein a protruding part with a flat anterior surface protruding forwards on the outer side of the top edge is formed at the tip of the needle cap.

18. The puncture method according to claim 17, wherein
the protruding part of the needle cap is formed protruding forwards from the base of the tip on the center line passing between the left and right as seen from the front of the tip, and
the outer surface of the scab removal part extends, from the protruding part, outwards around the circumference of the tip, with the top edge being formed at the farthest periphery of this outer surface.

19. The puncture method according to claim 18, wherein the scab removal part is formed on both the left and right sides of the protruding part.

20. The puncture method according to claim 15, wherein the scab removal part of the needle cap is formed on both left and right sides of the center line as seen from the front side of the tip, and the top edges on both sides are formed to be reverse to each other around the circumference of the tip.

21. The puncture method according to claim 15, wherein the inner surface of the needle cap curves convexly towards the outer surface.

22. The puncture method according to claim 14, wherein
a scab removal part is formed at the tip of the needle cap,
the scab removal part has a flat outer face parallel to the needle insertion axis and an inner surface located inside the outer face, and
a sharp top edge with a width in the direction of the needle insertion axis is formed by the outer face and the inner surface.

23. The puncture method according to claim 22, wherein a protruding part with a flat anterior surface protruding forwards on the outside of the sharp top edge is formed on the tip of the needle cap.

24. The puncture method according to claim 23, wherein
the protruding part of the needle cap is formed protruding forward from the base of the tip along the center line passing between the left and right as seen from the front side of the tip, and
the outer face of the scab removal part is formed at the side of the protruding part of the needle cap, and the top edge is formed at the periphery of the outer face furthest from the protruding part.

25. The puncture method according to claim 23, wherein the scab removal part is formed on both the left and right sides of the protruding part of the needle cap.

26. The puncture method according to claim 22, wherein the scab removal part is formed on both left and right sides of the center line as seen from the front side of the tip, and the top edges on both sides are formed on reverse sides each other to the left and right of the protruding part.

27. The puncture method according to claim 22, wherein the inner surface of the needle cap is curved convexly outwards.

28. The puncture method according to claim 15, wherein the inner surface tapers in the direction of the needle insertion axis so that the scab removal part grows thinner towards the front.

29. The puncture method according to claim 15, wherein the needle cap is used for a needle for puncturing during blood purification treatment.

30. The puncture method according to claim 15, wherein the needle cap is used for a needle for puncturing during drug administration or blood withdrawal.

31. A method of puncturing a shunt blood vessel with a puncture needle through a puncture hole formed by incision in the surface of a shunt blood vessel, wherein
the puncture needle is a needle having a tubular part with an angled end face angled with respect to the needle axis and a protruding part that protrudes forwards linearly from the tip of the angled end face of the tubular part, with the tip of the protruding part being narrower than the tubular part and being curved convexly in the front, and
the method of puncturing comprises a step of inserting the puncture needle in the shunt blood vessel through the puncture hole formed by incision starting with the protruding part.

32. A method of puncturing a shunt blood vessel, comprising a step of removing a scab formed at an entrance to a puncture route on a skin surface with a needle cap to be mounted on a puncture needle, and a step of puncturing the shunt blood vessel with the puncture needle after removal of the scab, wherein
the needle cap is a needle cap having a hollow holding part for holding the needle and a scab removal part on the tip of the holding part, with the scab removal part being formed from a circular arc-shaped outer surface curving around the needle insertion axis and an inner surface located inside the outer surface, and the needle cap further having a sharp top edge with a width in the direction of the needle insertion axis formed by the outer surface and inner surface, and
the scab is removed with the scab removal part of the needle cap.

33. A tubular or cylindrical incision needle for puncturing a shunt blood vessel and forming an incision on the surface of a shunt blood vessel, comprising an angled end face at the tip of the needle, wherein
the tip of the angled end face has a pointed extremity protruding apically from a hypothetical angled plane formed by cutting straight through the tip of the needle at an angle, and
the pointed extremity is curved towards the central axis of the needle,
cutting faces are formed on at least a part of the angled end face that includes the pointed extremity,
reverse cutting faces are formed on at least a part of the reverse surface of the angled end face that includes the pointed extremity, and
the foremost edge of the pointed extremity is formed by the cutting faces arranged symmetrically with the foremost edge therebetween, while the reverse cutting face is disposed not at the foremost edge, but arranged symmetrically with the center of the pointed extremity therebetween.

34. The incision needle according to claim 33, wherein the distance between the tip of the pointed extremity and the outer surface of the needle body is 6% to 50% of the outer diameter of the needle body.

35. A puncture needle for puncturing a shunt blood vessel through a puncture hole formed by incision in the surface of a shunt blood vessel, comprising:
a tubular part having an angled end face angled relative to the needle axis; and
a protruding part that protrudes forwards from the tip of the angled end face of the tubular part, wherein
the protruding part is narrower at the tip than the tubular part and has a convex curve at the front, and
the radius of the protruding part is 200 μm or more.

36. The puncture needle according to claim 35, wherein the curvature radius Re of the tip of the protruding part and the radius Ro of the outer diameter of the tubular part are in the relationship of 0.1×Ro≦Re<1.0×Ro.

37. A needle cap for removing a scab at the entrance to a puncture route in a skin surface, comprising: a hollow holding part for holding a needle; and a scab removal part at the tip of the holding part, wherein
the scab removal part is formed from a circular arc-shaped outer surface curving around the needle insertion axis and an inner surface located inside the outer surface, and has a sharp top edge that is formed from this outer surface and inner surface and has a width in the direction of the needle insertion axis, and
the scab removal part is formed substantially in a crescent shape in cross-section, and the angle formed by the arc of the outer surface and the arc of the inner surface at the top edge in this crescent cross-section is 5° to 89°.

38. The needle cap according to claim 37, wherein the length of the arc of the outer surface of the crescent cross-section is 5 to 50% of the maximum outer diameter of the holding part.

39. A blood purification instrument capable of constituting part of a blood purification circuit, comprising the needle cap according to claim 37, a needle inserted in this needle cap, a tube with this needle connected to a tip thereof, and a connecting part for connecting the posterior end of this tube to another tube.
